(19) United States
(12) Patent Application Publication
(10) Pub. No.: US 2014/0242157 A1
(43) Pub. Date: Aug. 28, 2014

(54) EPICATECHIN COMPOSITIONS AND METHODS

(71) Applicant: Cacao Bio-Technologies, LLC, Brooklyn, NY (US)

(72) Inventors: Daniel PRESTON, Brooklyn, NY (US); Randall B. MURPHY, Glenmoore, PA (US)

(73) Assignee: CACAO BIO-TECHNOLOGIES, LLC, Brooklyn, NY (US)

(21) Appl. No.: 14/269,767

(22) Filed: May 5, 2014

Related U.S. Application Data
(63) Continuation of application No. 13/070,916, filed on Mar. 24, 2011.
(60) Provisional application No. 61/317,528, filed on Mar. 25, 2010.
(60) Provisional application No. 61/317,540, filed on Mar. 25, 2010.
(60) Provisional application No. 61/175,561, filed on May 5, 2009.
(60) Provisional application No. 61/317,578, filed on Mar. 25, 2010.

(51) Int. Cl. A61K 36/185; A61K 45/06; A61K 9/14

(52) U.S. Cl. CPC A61K 36/185; A61K 9/14; A61K 45/06
USPC 424/450; 424/769; 424/499; 424/489; 424/43; 435/125

(57) ABSTRACT

Provided herein are methods and compositions for treating a subject who has pulmonary damage or an injury caused by or who is at risk for an injury caused by an anti-vesicant agent. The compositions comprise cacao extracts that include a mixture of epicatechin and one or more epicatechin oligomers and a pharmaceutically acceptable carrier. Also provided are methods for fermenting cacao to enhance epicatechin and antioxidant content.

### RELATED APPLICATIONS

This application is a Continuation of co-pending U.S. patent application Ser. No. 13/070,916, filed Mar. 24, 2011, which claims the benefit of the filing date of U.S. Provisional Application Nos. 61/317,528, 61/317,540, 61/317,561, and 61/317,578, which were filed on Mar. 25, 2010. For the purpose of any U.S. application that may claim the benefit of U.S. Provisional Application Nos. 61/317,528, 61/317,540, 61/317,561, and 61/317,578, the contents of these earlier filed applications are hereby incorporated by reference in their entirety.

### FIELD OF THE INVENTION

The present invention relates to compositions and methods for treatment of pulmonary damage and damage caused by vesicants in a subject. The compositions include epcatechins and epicatechin oligomers. Also provided are fermentation methods for cacao to enhance epicatechin and antioxidant content.

### BACKGROUND

Mammalian inflammatory pathways are an important consequence of the immune system and play a vital role in the normal homeostasis of the body. Whilst short-term inflammation has a protective function, in chronic diseases such as arthritis and asthma, inflammation is associated with the typical oedema, swelling, pain and organ dysfunction. Inflammation is also associated with injuries caused by anti-vesicant agents. There is a continuing need for methods and compositions that reduce inflammation.

### SUMMARY

In addition to being potent antioxidants, epcatechins and epicatechin oligomers are extremely reactive with activated sulfide or sulfhydryl groups. Simple treatment of epicatechin oligomers with benzyl mercaptan results in degradation of the polymer to monomeric epithiocatechin units. In the case of mustard gas, the reaction is instantaneous.

The presence of epicatechin oligomers in this invention is highly desired because there are many possible reactive groups per mole of the epicatechin oligomer. A relatively small amount of high molecular mass epicatechin oligomers will therefore provide protection to skin against the alkylating activity of mustard gas, phosgene oxime, and related agents.

The protectant or decontaminant may be clothing, combat gear, a protective shelter, a weapon, a piece of equipment, a filter, a sponge, a foam, a spray, a lotion, or a gas. The protectant or decontaminant may be used to prevent exposure of a subject to a vesicant agent. The protectant may be used to treat a subject exposed to a vesicant agent or treat an injury induced by a vesicant agent. The decontaminant may be used to decontaminate a subject or an object exposed to a vesicant agent. In some embodiments, the protectant or decontaminant further comprises a second antivesicant compound, a supplementary active compound, or both. In some embodiments, the present invention provides a kit comprising the protectant or decontaminant and instructions for use.

In some embodiments, the present invention relates to a method of decontaminating an area exposed to a vesicant agent comprising contacting a compound having the structural formula

In some embodiments, the present invention provides a kit for treating, preventing, or inhibiting an injury induced by a vesicant agent comprising at least one compound having the structural formula corresponding to a catechin, epicatechin, or epicatechin oligomer.

In some embodiments, the kit further comprises a second antivesicant compound such as L-cysteine. In some embodiments, the kit further comprises a supplementary active compound such as an anti-inflammatory or an anti-protease drug or compound.

In some embodiments, the present invention is directed to a kit for decontaminating an area exposed to a vesicant agent comprising at least one compound having the structural formula corresponding to a catechin, epicatechin, or epicatechin oligomer

The present invention makes use of the combined anti-inflammatory properties and antioxidant properties of epicatechins and epicatechin oligomers. More specifically the instant invention utilizes the properties of mixtures of epicatechins and epicatechin oligomers in the form that they are present as natural products as extracted from plant sources rather than as highly purified synthetic compounds, known to those normally skilled in the art as natural products. Even more specifically, the said epicatechins and epicatechin oligomers are in the form in which they are obtained by extraction from seeds of the cacao tree Theobroma cacao.

Most specifically, the present invention relates to the synergistic effect of combined cacao epicatechins and epicatechin oligomers upon asthma, chronic obstructive pulmonary disease and rheumatoid and osteoarthritis, and other inflammatory conditions.

Mammalian inflammatory pathways are an important consequence of the immune system and play a vital role in the normal homeostasis of the body. Whilst short-term inflammation has a protective function, in chronic diseases such as arthritis and asthma, inflammation is associated with the typical oedema, swelling, pain and organ dysfunction.

Prostaglandins and leukotrienes are potent biologically active structures that normally play an essential role in tissue homeostasis. However, following cellular injury or trauma the respective production of specific prostaglandins and leukotrienes shifts to an inflammatory reaction with local physiological effects

Mammalian inflammatory pathways are an important consequence of the immune system and play a vital role in the normal homeostasis of the body. Whilst short-term inflammation has a protective function, in chronic diseases such as arthritis and asthma, inflammation is associated with the typical oedema, swelling, pain and organ dysfunction.

Prostaglandins and leukotrienes are potent biologically active structures that normally play an essential role in tissue homeostasis. However, following cellular injury or trauma the respective production of specific prostaglandins and leukotrienes shifts to an inflammatory reaction with local physiological effects

The details of one or more embodiments of the invention are set forth in the accompanying drawings and the description below. Other features, objects, and advantages of the invention will be apparent from the description and drawings, and from the claims.

### DETAILED DESCRIPTION

### 1. General

Chocolate, cocoa butter, and cocoa-flavoring ingredients are derived from the tropical fruit Theobroma cacao. Cocoa is ingested by many cultures and the discovery of its residue in ancient Mayan vessels suggests that humans have been consuming it, in some form, since at least 480 A.D. Common components of fresh cocoa beans (cotyledons) include theobromine, caffeine, flavinoid polyphenols, and saturated and monounsaturated fatty acids.

Flavonoids are a major class of plant polyphenolics, which comprises thousands of compounds such as flavonols, flavones, flavanones, flavanols, anthocyanins, dihydroflavonols, isoflavones and chalcones. Flavonoids are widely distributed in the plant kingdom, being present in a broad range of commonly consumed fruits and vegetables and plant-derived products such as cocoa, tea, and wine. Flavonols like quercetin mostly occur in foodstuffs as glycosides and, in general, the first step in their metabolism is likely to be deglycosylation before absorption in the small intestine; nonetheless they are generally well absorbed in man as well as in animals.

These beneficial actions of the flavinoids are due in part to their antioxidant activity. Antioxidant components are microconstituents present in the diet that can delay or inhibit lipid oxidation, by inhibiting the initiation or propagation of oxidizing chain reactions, and are also involved in scavenging free radicals. Food such as fruits, vegetables and grains are reported to contain a wide variety of antioxidant components, including phenolic compounds. These compounds are found to be well correlated with antioxidant potential

The interest in flavonoids has grown in the last fifteen years after the publication of several epidemiological studies showing an inverse correlation between dietary consumption of flavonoid-rich products and reduced incidence and mortality from cardiovascular disease and cancer Specifically epicatechins, such as epicatechin gallates originally identified in tea, have been reported to possess antimutagenic, antibacterial, antioxidant, antitumor and cancer preventive properties. Certain actions may also depend on pharmacological activities beyond their antioxidant properties. For example, tea polyphenols may induce apoptosis and are known to inhibit the growth of several cancer cell lines Polyphenols from other plant sources also inhibit the cellular expression of interleukin-8 and monocyte chemoattractant-1 when induced by the pro-inflammatory cytokine, tumor necrosis factor, and modulate of the pro-inflammatory cytokine interleukin-1 Epicatechin derivatives have also been shown to have antiviral and antibacterial activities.

Cacao products are rich in polyphenols such as epicatechin oligomers, as well as in other catechins and procyanidins. It has been reported that chocolate is a major source of catechins 60% of the total phenolics in raw cocoa beans are flavanol monomers (epicatechin and catechin) and procyanidin oligomers (dimer to decamer). These compounds are well known in the prior art to combat free radicals, which are harmful to the human and animal body. Free radicals cause degenerative human diseases such as cancer, heart disease, and cerebrovascular disease through multiple mechanisms. In vitro studies demonstrated that the cacao flavinoid compounds have several biological activities, such as the ability to scavenge superoxide radicals and hydroxyl radicals, reduce lipid peroxyl radicals and inhibit lipid peroxidation Epicatechin oligomers in chocolate and cocoa are orally well absorbed and are metabolized and excreted as various conjugates. In a clinical study, cocoa powder supplementation was found to delay the oxidation of low-density lipoprotein.

### 2. Epicatechins

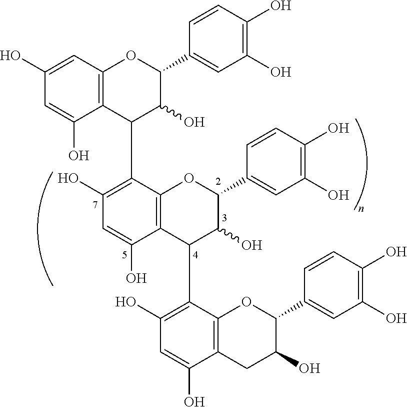 [SMILES: Oc1cc(O)c2c(c1)O[C@H](c1ccc(O)c(O)c1)C(O)C2c1c(O)cc(O)c2c1O[C@H](c1ccc(O)c(O)c1)C(O)C2c1c(O)cc(O)c2c1O[C@H](c1ccc(O)c(O)c1)[C@@H](O)C2]

I Epicatechins represent the basic monomeric unit of the proanthrocyanodins

Proanthocyandins or so-called, condensed tannins; are polymers made of flavan-3-ol units linked together by carbon-carbon bonds. They are the most widespread polyphenols in plants after lignins and can be found in leaves, fruits, woods, barks, or roots, often in high concentration (over 50% in some barks). Many food products contain proanthrocyandins but cacoa is amongst the highest content of any known material. See for example Ortega, N., M. P. Romero, et al. (2008). “Obtention and characterization of phenolic extracts from different cocoa sources.” Journal of Agricultural & Food Chemistry 56(20): 9621-7.; Kim, H. and P. G. Keeney (1984). “(−)-Epicatechin content in fermented and unfermented cocoa beans.” Journal of Food Science 49(4): 1090-1092.; Kofink, M., M. Papagiannopoulos, et al. (2007). “(−)-Catechin in cocoa and chocolate: occurrence and analysis of an atypical flavan-3-ol enantiomer.” Molecules 12(7): 1274-88.; Gu, L., S. E. House, et al. (2006). “Procyanidin and catechin contents and antioxidant capacity of cocoa and chocolate products.” Journal of Agricultural & Food Chemistry 54(11): 4057-61.; Jalil, A. M. and A. Ismail (2008). “Polyphenols in cocoa and cocoa products: is there a link between antioxidant properties and health?” Molecules 13(9): 2190-219 which are herein incorporated by reference.

The basic molecular epicatechin unit is:

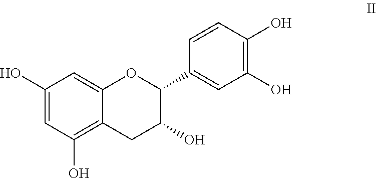 [SMILES: Oc1cc(O)c2c(c1)O[C@H](c1ccc(O)c(O)c1)[C@H](O)C2]

typically it is present in the free from in cocao as the gallate ester derivative:

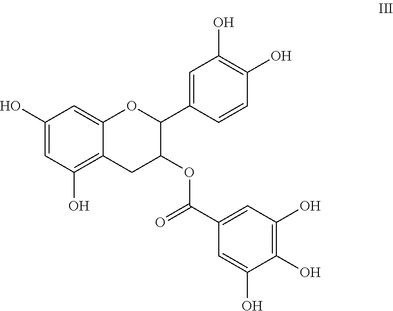 [SMILES: O=C(OC1Cc2c(O)cc(O)cc2OC1c1ccc(O)c(O)c1)c1cc(O)c(O)c(O)c1]

which has in itself been shown in prior art to be an extremely potent antioxidant material.

### 3. Health Benefits of Epicatechin Oligomers

As mentioned supra the epicatechin oligomers present in cacoa are known to produce many health benefits. See for example Allen, R. R., L. Carson, et al. (2008). “Daily consumption of a dark chocolate containing flavanols and added sterol esters affects cardiovascular risk factors in a normotensive population with elevated cholesterol.” Journal of Nutrition 138(4): 725-31.; Bahadorani, S, and A. J. Hilliker (2008). “Cocoa confers life span extension in Drosophila melanogaster.” Nutrition Research 28(6): 377-82; Barnard, K. (2006). “Flavanols, cocoa, and cardiology.” Circulation 114(23): f191-2.; Bisson, J. F., M. A. Guardia-Llorens, et al. (2008). “Protective effect of Acticoa powder, a cocoa polyphenolic extract, on prostate carcinogenesis in Wistar-Unilever rats.” European Journal of Cancer Prevention 17(1): 54-61.; Bisson, J. F., S. Hidalgo, et al. (2007). “Preventive effects of ACTICOA powder, a cocoa polyphenolic extract, on experimentally induced prostate hyperplasia in Wistar-Unilever rats.” Journal of Medicinal Food 10(4): 622-7.; Bisson, J. F., S. Hidalgo, et al. (2007). “Therapeutic effect of ACTICOA powder, a cocoa polyphenolic extract, on experimentally induced prostate hyperplasia in Wistar-Unilever rats.” Journal of Medicinal Food 10(4): 628-35.; Bisson, J. F., A. Nejdi, et al. (2008). “Effects of long-term administration of a cocoa polyphenolic extract (Acticoa powder) on cognitive performances in aged rats.” British Journal of Nutrition 100(1): 94-101.; Chen, D. M., X. Cai, et al. (2006). “Inhibitory effects of procyanidin B(2) dimer on lipid-laden macrophage formation.” Journal of Cardiovascular Pharmacology 48(2): 54-70.; Cho, E. S., K. W. Lee, et al. (2008). “Cocoa procyanidins protect PC12 cells from hydrogen-peroxide-induced apoptosis by inhibiting activation of p38 MAPK and JNK.” Mutation Research 640(1-2): 123-30.

Cohen, D. L. and R. R. Townsend (2007). “Cocoa ingestion and hypertension-another cup please?” Journal of Clinical Hypertension 9(8): 647-8.; Cooper, K. A., J. L. Donovan, et al. (2008). “Cocoa and health: a decade of research.” British Journal of Nutrition 99(1): 1-11.; Corti, R., A. J. Flammer, et al. (2009). “Cocoa and cardiovascular health.” Circulation 119(10): 1433-41.; Davison, K., A. M. Coates, et al. (2008). “Effect of cocoa flavanols and exercise on cardiometabolic risk factors in overweight and obese subjects.” International Journal of Obesity 32(8): 1289-96.; Dinges, D. F. (2006). “Cocoa flavanols, cerebral blood flow, cognition, and health: going forward.[comment].” Journal of Cardiovascular Pharmacology 47 Suppl 2: 5221-3.; Engler, M. B. and M. M. Engler (2006). “The emerging role of flavonoid-rich cocoa and chocolate in cardiovascular health and disease.” Nutrition Reviews 64(3): 109-18.; Erdman, J. W., Jr., L. Carson, et al. (2008). “Effects of cocoa flavanols on risk factors for cardiovascular disease.” Asia Pacific Journal of Clinical Nutrition 17 Suppl 1: 284-7.; Erlejman, A. G., C. G. Fraga, et al. (2006). “Procyanidins protect Caco-2 cells from bile acid- and oxidant-induced damage.” Free Radical Biology & Medicine 41(8): 1247-56.; Esquenazi, D., M. D. Wigg, et al. (2002). “Antimicrobial and antiviral activities of polyphenolics from Cocos nucifera Linn. (Palmae) husk fiber extract.” Research in Microbiology 153(10): 647-652.; Faridi, Z., V. Y. Njike, et al. (2008). “Acute dark chocolate and cocoa ingestion and endothelial function: a randomized controlled crossover trial.” American Journal of Clinical Nutrition 88(1): 58-63.; Farouque, H. M., M. Leung, et al. (2006). “Acute and chronic effects of flavanol-rich cocoa on vascular function in subjects with coronary artery disease: a randomized double-blind placebo-controlled study.” Clinical Science 111(1): 71-80.; Ferri, C., D. Grassi, et al. (2006). “Cocoa beans, endothelial function and aging: an unexpected friendship” Journal of Hypertension 24(8): 1471-4. which are incorporated herein by reference.

Unfortunately, the manufacturing process dramatically influences the concentration of epicatechin oligomers and antioxidant tannins, and most commercially produced cocoa contains little of other valuable antioxidants. Commercial cocoa products are generally produced through an alkali treatment process. This process is known to remove the majority of epicatechin oligomers. Indeed studies of the antioxidant activity of chocolate indicate that more than 90% of the beneficial epicatechin oligomers, procyanidins, and catechins are eliminated by this treatment. See for example Andres-Lacueva, C., M. Monagas, et al. (2008). “Flavanol and flavonol contents of cocoa powder products: influence of the manufacturing process.” Journal of Agricultural & Food Chemistry 56(9): 3111-7.; Oliviero, T., E. Capuano, et al. (2009). “Influence of roasting on the antioxidant activity and HMF formation of a cocoa bean model systems.” Journal of Agricultural & Food Chemistry 57(1): 147-52.; Summa, C., J. McCourt, et al. (2008). “Radical scavenging activity, anti-bacterial and mutagenic effects of cocoa bean Maillard reaction products with degree of roasting.” Molecular Nutrition & Food Research 52(3): 342-51. Even so, it has been shown that on a per-serving basis a cup of alkali processed cocoa has antioxidant activities which are 4-5 times stronger than that of black tea, 2 3 times stronger than green tea, and almost 2 times stronger than red wine.

Considerable effort has been expended to develop cocoa products with increased epicatechin content, see for example Tomas-Barberan, F. A., E. Cienfuegos-Jovellanos, et al. (2007). “A new process to develop a cocoa powder with higher flavinoid monomer content and enhanced bioavailability in healthy humans.” Journal of Agricultural & Food Chemistry 55(10): 3926-35. A series of patents by the group at Mars, Inc. and Hershey, Inc. has focused upon this effort. See for example 6905715, Jun. 14, 2005 Kealey, Kirk S., COCOA SOLIDS HAVING A HIGH COCOA PROCYANIDIN CONTENT; 6887501, May 3, 2005, Kealey, Kirk S. ALKALIZED COCOA SOLIDS; 6737088, May 18, 2004, Kealey, Kirk S. COCOA EXTRACTS PREPARED FROM COCOA SOLIDS HAVING HIGH COCOA POLYPHENOL CONTENTS; 6673379, Jan. 6, 2004 Kealey, Kirk S., USE OF NON-ALKALIZED COCOA SOLIDS IN A DRINK; 6558713, May 6, 2003 Kealey, Kirk S., HEALTH OF A MAMMAL BY ADMINISTERING A COMPOSITION CONTAINING AT LEAST ONE COCOA POLYPHENOL INGREDIENT; 6372267, Apr. 16, 2002, Kealey, Kirk S. FOODS CONTAINING COCOA SOLIDS HAVING HIGH COCOA POLYPHENOL CONTENTS; 6015913, Jan. 18, 2000 Kealey, Kirk S., METHOD FOR PRODUCING FAT AND/OR SOLIDS FROM COCOA BEANS; 6312753, Nov. 6, 2001 Schmitz, Harold H., COCOA COMPONENTS, EDIBLE PRODUCTS HAVING ENRICHED POLYPHENOL CONTENT, METHODS OF MAKING SAME AND MEDICAL USES; 08157039 Dec. 24, 2008 ANDERSON, Brent, A. EDIBLE PRODUCTS HAVING A HIGH COCOA POLYPHENOL CONTENT AND IMPROVED FLAVOR AND THE MILLED COCOA EXTRACTS USED THEREIN; 05123096 Dec. 29, 2005 SCHMITZ, Harold, H. COMPOSITIONS AND METHODS OF USE OF DIMER DIGALLATES;

05072726 Aug. 11, 2005 SCHMITZ, Harold, H. COMPOSITIONS AND METHODS OF USE OF A-TYPE PROCYANIDINS; 01093690 Dec. 13, 2001 HAMMERSTONE, JOHN, F., Jr. AN IMPROVED METHOD FOR EXTRACTING COCOA PROCYANIDINS; 97036597 Oct. 9, 1997 ROMANCZYK, Leo, J., COCOA EXTRACT COMPOUNDS AND METHODS FOR MAKING AND USING THE SAME, which are herein incorporated by reference. However, these involve methods, processes, and compositions which are complex and difficult to prepare, in many cases involving multiple steps of extraction.

### DEFINITIONS

As used herein, the term “epicatechin oligomers” encompasses the compounds of the structure:

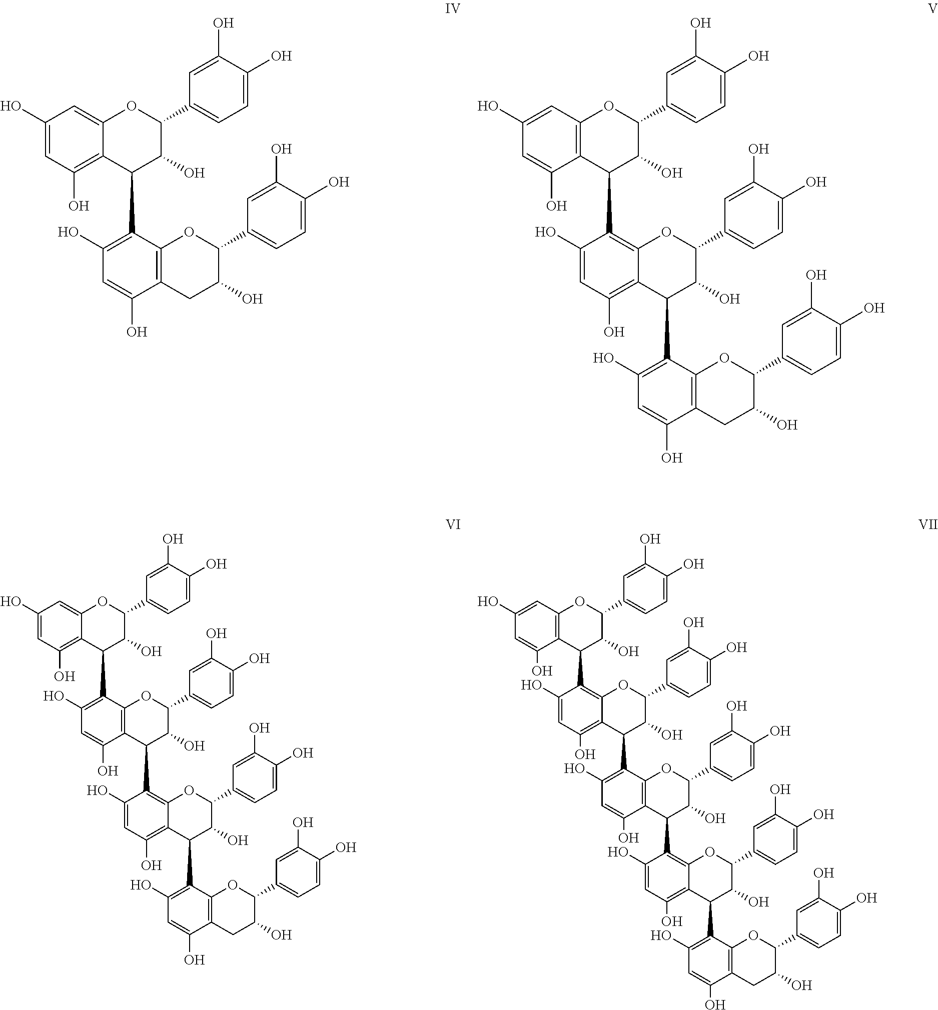

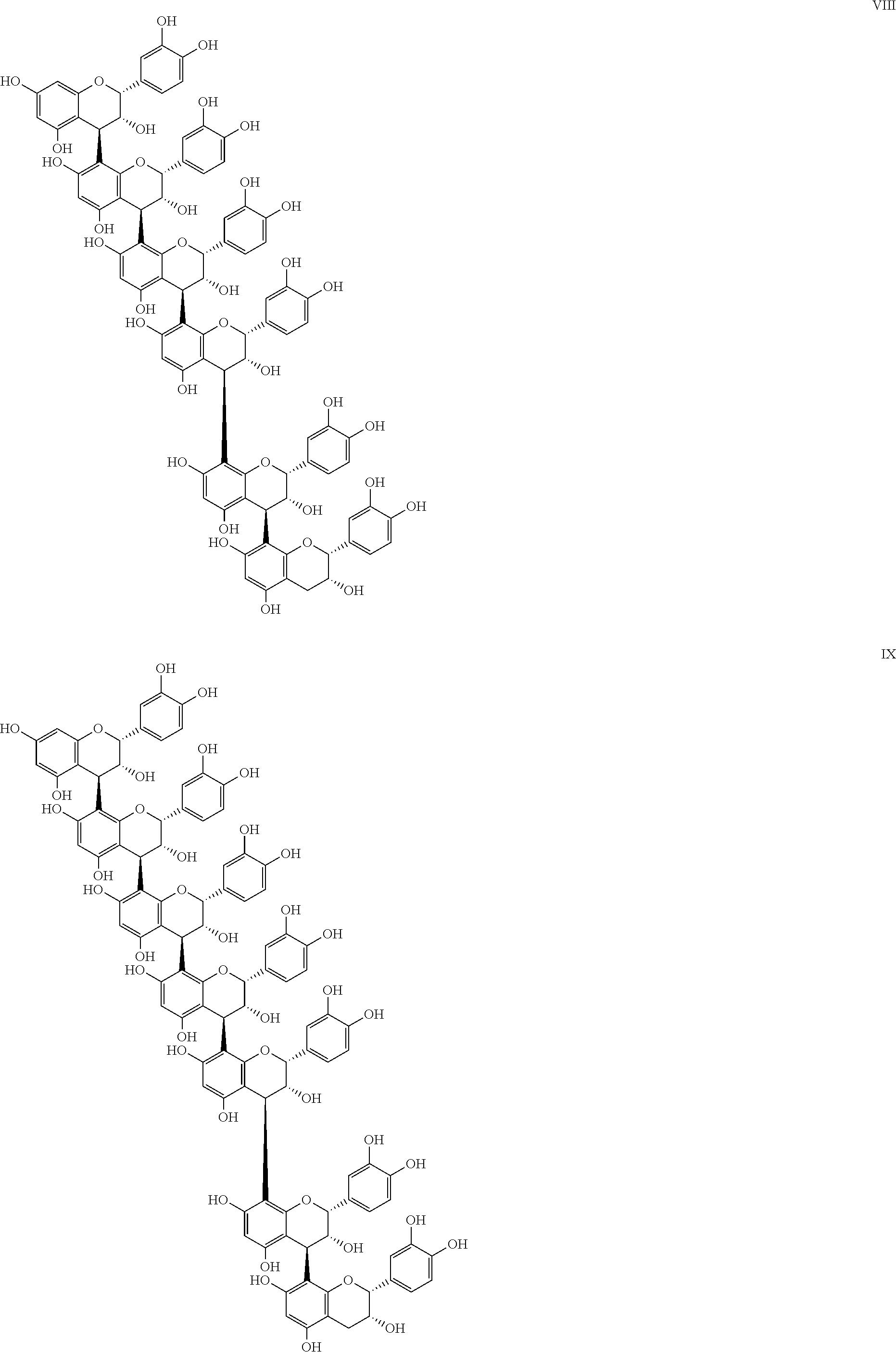

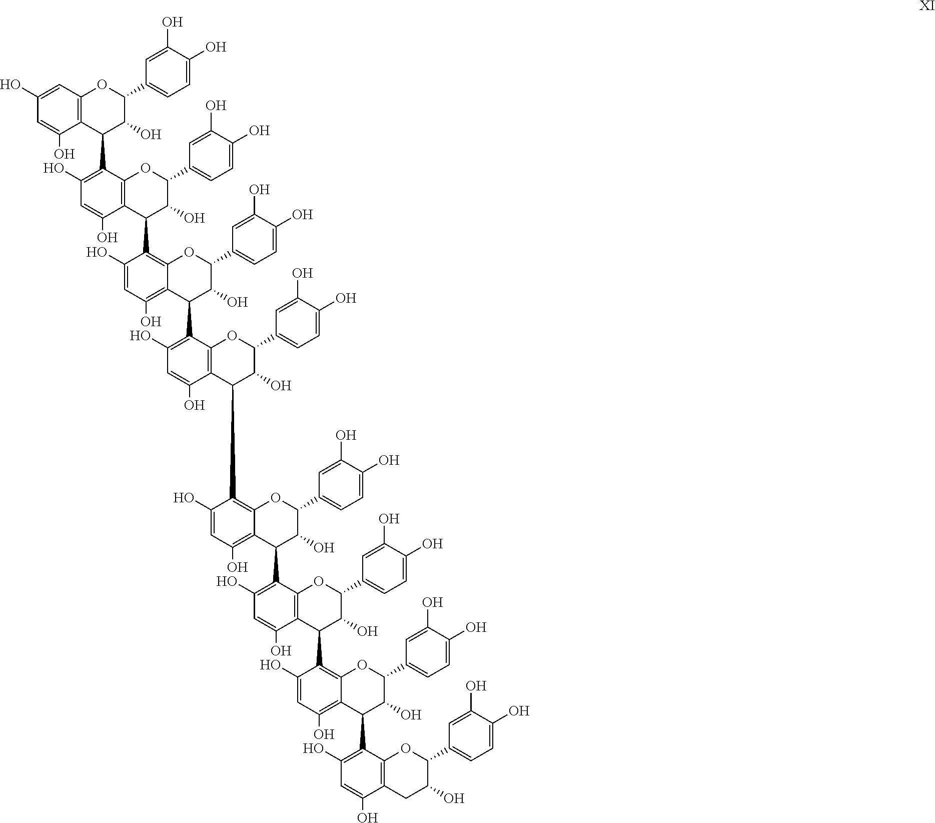

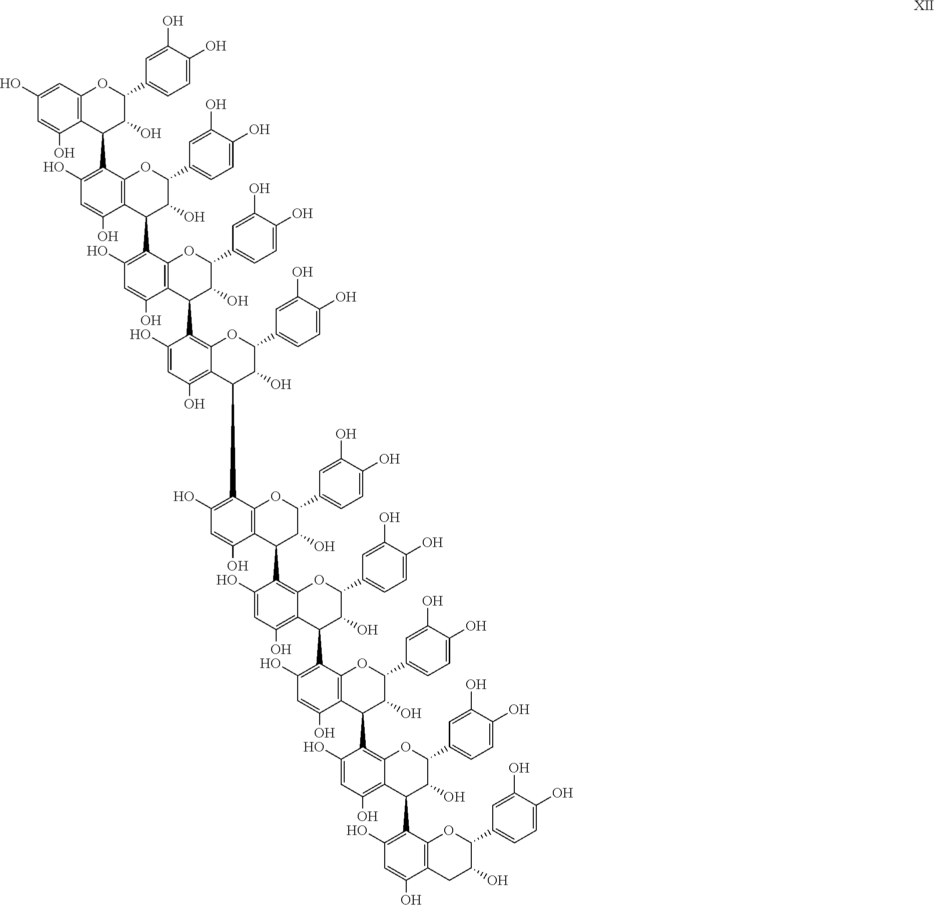

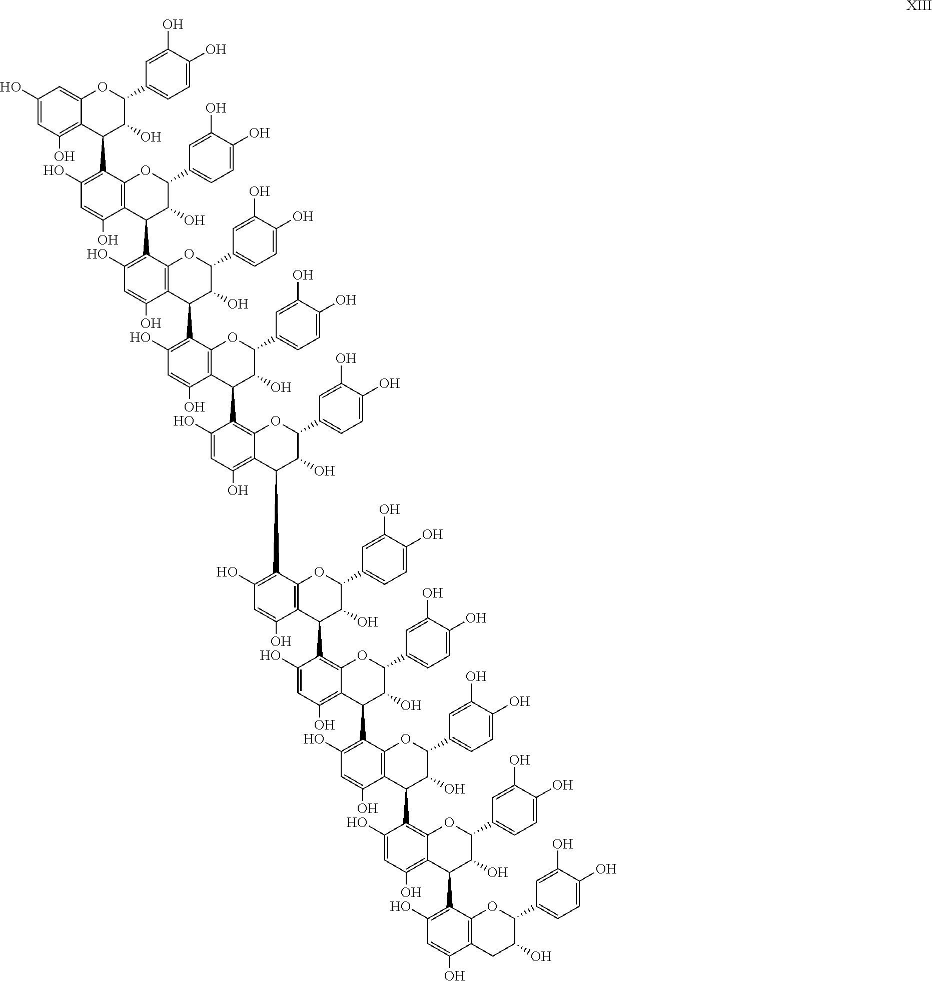

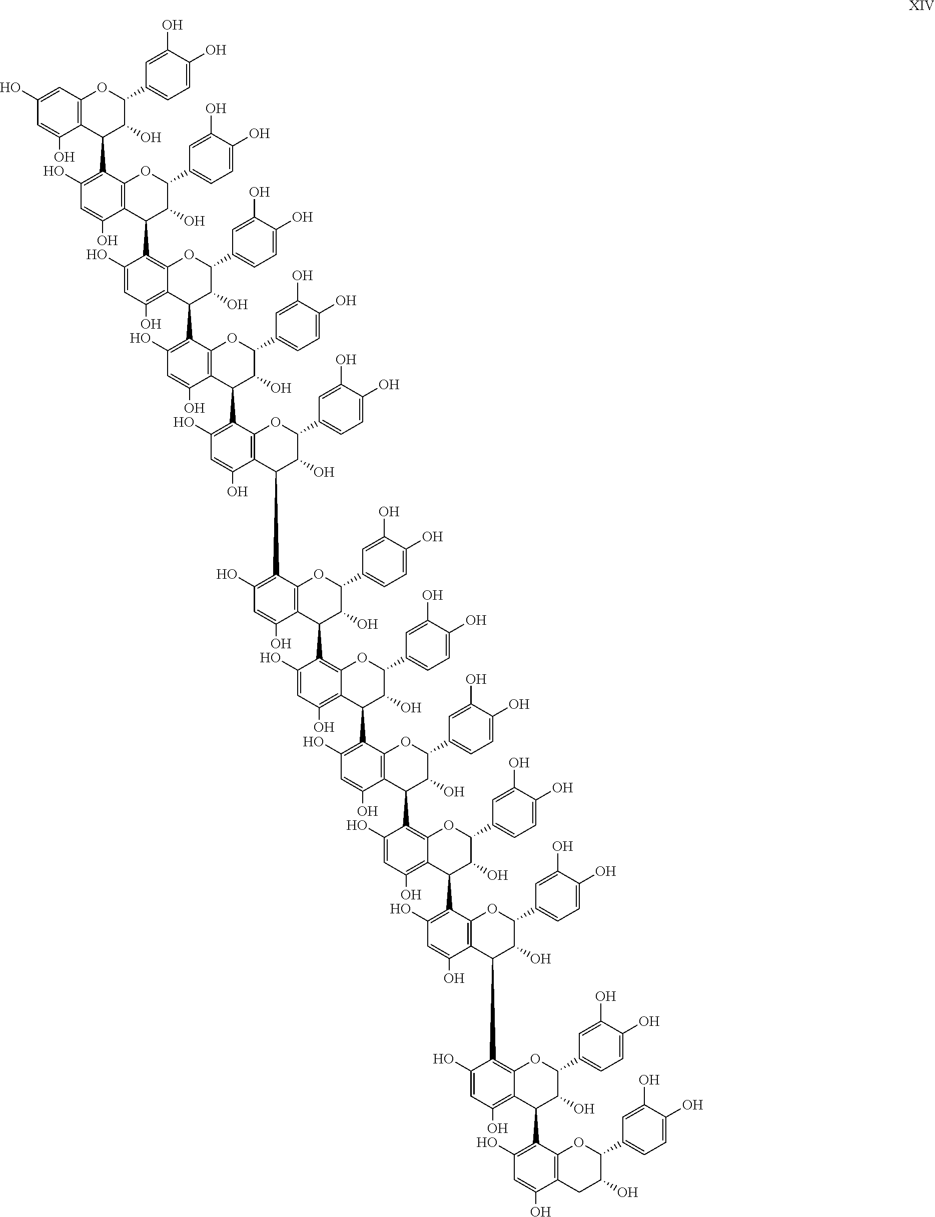

This also includes epigallocatechin anlogues of the compounds IV-XIV supra, exemplified by in a nonlimiting manner XXV-XXI below:

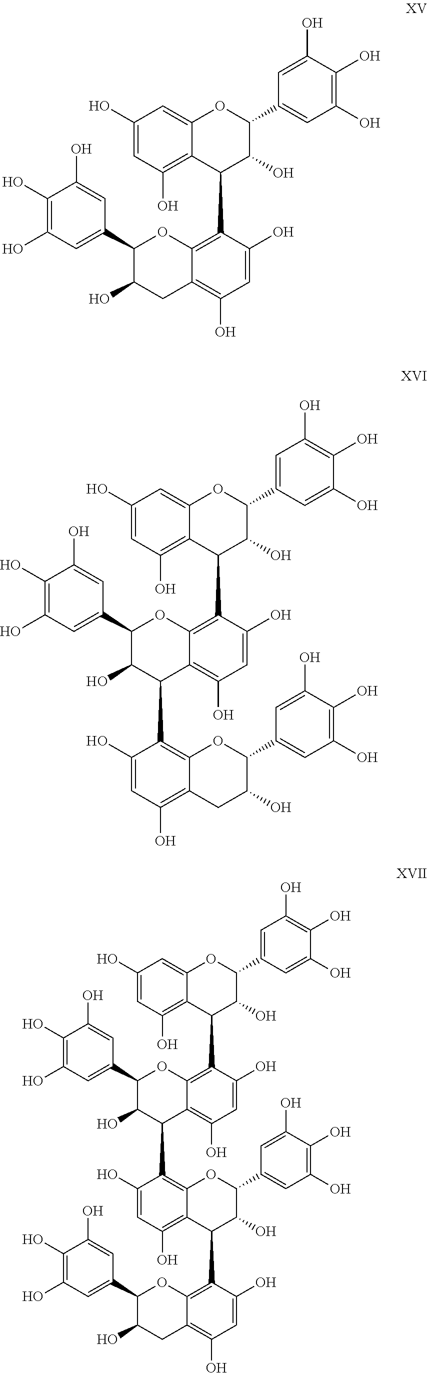

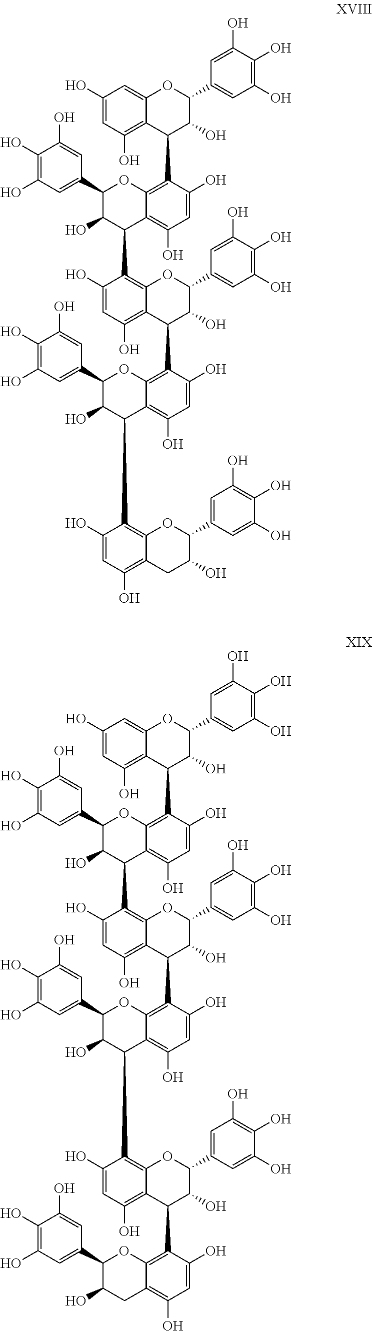

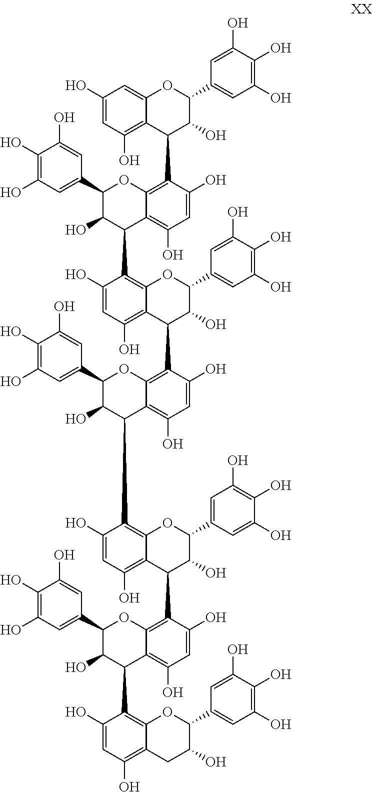

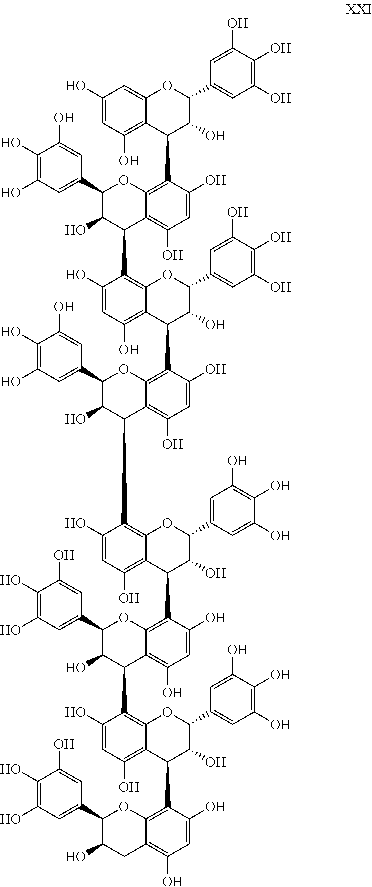

It should be noted that oligomers up to the range of dodecamers (N=12) are present in extracts of Theobroma Cacao which have not been subjected to degradative, alkaline conditions. Accordingly, the examples of oligomers illustrated here are nonlimiting with regard to the number of monomers, given that most commonly the number of said oligomers most commonly is in the range of 3 to 6, but can also be significant in the range of N=6 to N=12.

As used herein, the term “alkyl” encompasses linear or branched structures and combination thereof, having the indicated number of carbon atoms. Thus, for example, C₍₁₋₆₎alkyl includes methyl, ethyl, propyl, 2-propyl, s- and t-butyl, butyl, pentyl, hexyl, 1.1-dimethylethyl, cyclopropyl, cyclobutyl, cyclopentyl and cyclohexyl.

As used herein the compounds of the invention may have one or more asymmetric centers. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all such isomeric forms of the epicatechin oligomers supra. Some formulae are shown above without a definite stereochemistry at certain positions. The present invention includes all stereoisomers of the epicatechin oligomers and pharmaceutically acceptable salts thereof.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration. If desired, racemic mixtures of the compounds may be separated so that the individual enantiomers or diastereomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art. Alternatively, any enantiomer or diastereomer of a compound may be obtained by stereoselective synthesis using optically pure starting materials or reagents of known configuration by methods well known in the art.

The term “pharmaceutically acceptable” means that the carrier, diluent or excipient must be compatible with the other ingredients of the formulation and not deleterious to the recipient thereof.

The terms “administration of or “administering a” compound should be understood to mean providing a compound of the invention to the individual in need of treatment in a form that can be introduced into that individual's body or topically into the individual's dermis in a therapeutically useful form and therapeutically useful amount.

The terms “effective amount” or “therapeutically effective amount” means the amount of the subject compound that will elicit the biological or medical response of a tissue, system, animal or human that is being sought by the researcher, veterinarian, medical doctor or other clinician.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

As used herein, the term “pharmaceutically acceptable salts” encompasses both the metallic (inorganic) salts and organic salts; a list of which is given in Remington's Pharmaceutical Sciences, 17th Edition, pg. 1418 (1985). It is well known to one skilled in the art that an appropriate salt form is chosen based on physical and chemical stability, flowability, hydroscopicity and solubility. As will be understood by those skilled in the art, pharmaceutically acceptable salts include, but are not limited to salts of inorganic acids such as hydrochloride, sulfate, phosphate, diphosphate, hydrobromide, and nitrate or salts of an organic acid such as malate, maleate, fumarate, tartrate, succinate, citrate, acetate, lactate, methanesulfonate, p-toluenesulfonate or pamoate, salicylate and stearate. Similarly pharmaceutically acceptable cations include, but are not limited to sodium, potassium, calcium, aluminum, lithium and ammonium (especially ammonium salts with secondary amines). salts may also be obtained with bases such as ammonium hydroxide or secondary or tertiary amines (such as diethylamine, triethylamine, piperidine, piperazine, morpholine) or with basic amino-acids, or with osamines (such as meglumine) or with amino-alcohols (such as 3-aminobutanol and 2-aminoethanol. Preferred salts of this invention include potassium, sodium, calcium and ammonium salts. Salts in the solid form may exist in more than one crystal structure, and may also be in the form of hydrates

As used herein, the term “pharmaceutically acceptable esters” encompasses esters using alkyl alcohols

The invention provides pharmaceutical or veterinary composition comprising an epicatechin in combination with epicatechin oligomers, or a pharmaceutically or veterinarily acceptable derivative thereof. The epicatechin or epicatechin oligomers can be formulated with other flavinoids. Nonlimiting examples of flavinoids preferred in the present formulation are quercetin, rutin, kaempferol, myricetin, isorhamnetin, apigenin, luteolin, hesperetin, naringenin, eriodictyol, a catechin, an epicatechin, a theaflavin, a thearubigin, cyanidin, delphinidin, malvidin, pelargonidin, peonidin, or petunidin. Mixtures of any two or more of these or other flavonoids may be used.
Preparation of Extracts from Theobroma cacao Enriched in Epicatechin Oligomers

Any art-known method that yields extracts from Theobroma cacao that are enriched in epicatechin oligomers can be used. An exemplary method for preparation of extracts from Theobroma cacao enriched in epicatechin oligomers is as follows. A 50-kilogram sample of cocoa Nibs obtained from a Criollo strain (Rizek, S. A., Republica Dominicana) which has been substantially freed from cacao hulls is reduced to a powder between 250-325 mesh with the aid of a Wiley mill, taking care to cool the mill with the aid of liquid nitrogen to prevent overheating which will lead to chemical alteration and deterioration of the desired epicatechin oligomers. All subsequent procedures described herein are optionally carried out in the presence of Argon gas, in order to avoid oxidation and decomposition of the desired epicatechin oligomers. Most subsequent processing is carried out with the aid of a 50-pliter rotary evaorator (Buchi AG, Switzerland) and a flanged glass flask designed to mate with said evaporator. Into this flanged flask is placed the cocoa nibs, and the flask is emplaced on the rotary evaporator and dried at 45-50° C. with the aid of a thermoregulated water bath and an Edwards rough pump maintaining ca. 1 torr pressure. The rotary evaporator is cooled with an ethylene glycol bath maintained at between −15 to −25° C. At the end of this time, the flask is removed and into it is emplaced 2 volumes of hexane (Baker Analytical reagent grade). This is then placed back upon the rotary evaporator and warmed at 30° C. under slight argon pressure overnight without cooling of the rotary evaporator. The hexane has previously been sparged with argon. It has been determined that heptane can also be used in this procedure, although it is slightly more expensive but less toxic to work with. The hexane layer after the extraction period is decanted and retained and the extracted nibs are then extracted with a second portion of 2-3 volumes of fresh, argon-sparged hexane and left overnight as described supra. The hexane fraction is again decanted, hexane fractions are combined, and the hexane fractions extracted to dryness to yield desired lipids. The hexane is recovered and can be reused repeatedly until it has accumulated a large amount of cacao volatiles (4-6 extractions). At this time the hexane can be purified for reuse by passage through a large metal column (6 inches diameter by 4 feet long) coarsely backed with 25-40 mesh activated charcoal ((W.R. Grace), or if desired can be incinerated. The delipidated cacao nibs are dried in a stream of argon and are extracted with 3 volumes of methanol water (90:10) which has been sparged with argon, and is refluxed overnight with the aid of an efficient reflux condenser and a heating mantle under argon. The supernatant is set aside, and a second extraction overnight under reflux is performed. The supernatants are combined, and the extracted nibs are retained for other possible uses, such as the manufacture of epithiocatechins. The combined supernatants are reduced under dryness to provide a dry, powdered material closely resembling freeze-dried coffee, which is termed EXTRACT 1.

One method to enrich EXTRACT 1 in epicatechin oligomers and that can be used on an industrial scale but does not require high efficiency liquid chromatography is the following. A 100 gm sample of Extract 1 is dissolved in 2 L of 20 mM potassium phosphate buffer, pH 8.2. Warming may be required to effect this step. Into this is added 500 gm of DE-52 cellulose (Whatman, UK) and this is stirred gently with the aid of a mechanical (not magnetic) stirred at 40 C for 1-2 hours, under argon. At the end of this time the material is filtered on a Buchner funnel and washed with deionized water. The supernatant and washings are discarded and the cellulose and epicatechin oligomers are extracted batchwise with increasing concentrations of NaCl of 100 mM, 300 mM, 500 mM, and 1 M (3 liter portions), These salt solutions all are prepared in 50 mM phosphate buffer, pH 8.2. These are then back-extracted using continuous extraction into isobutyl acetate. The isobutyl acetate extracts are dried using a conventional laboratory scale buchi rotary evaporator. Mass spectrometric analysis of these extracts demonstrates that the higher salt extracts contain an enriched proportion of epicatechin oligomers.

Optionally, the extraction can be carried out with other sorbents, such as SP-1 (Supelco, Altoona Pa.) in a similar manner, with slightly different results in terms of the concentration of the epicatechin oligomers in the fractions. In some experiments, it has also been found useful to use a small concentration of an organic solvent, typically methyl, ethyl, isopropyl, or n-propyl alcohol, in the extracting buffers, from the range of 1% (v/v) to 10% (v/v), or n-butyl or 2-butyl alcohol in the range of 0.1-1.5% (v/v).

Further, it has been found that the use of conventional chromatographic sorbents, particularly alumina, leads to poor results and recovery due to chemical transformation of the epicatechin oligomers on the column catalyzed by the silica and alumina to undesired products, even under argon. The use of large amounts of organic solvents is similarly disadvantageous. In the method described above, the organics which are used can be recycled and the aqueous extractions of the cellulose and the solid phase sorbed are relatively “green” in character,

### Antivesicants

Generally, the present invention provides epicatechom amd epicatechin oligomers for treating, preventing, or inhibiting injuries induced by vesicant agents which includes sulfur mustard (bis-2-chloroethylsulfide (HD)), Nitrogen mustard (Mustargen7), Lewisite, phosgene oxime, and combinations thereof. Preferably, the present invention epicatechin and epicatechin oligomern containing compounds for treating, preventing, or inhibiting bis-2-chloroethylsulfide (HD) induced injuries.

The antivesicant activities of epicatechin compounds were assessed by the Mouse Ear Antivesicant Drug Screening Assay Protocol For the pre-treatment experiments, given concentrations, based on toxicity expectations, limits of solubility, or both, of the test compounds were applied to one side of the mouse ear 15 minutes prior to the HD exposure. For the treatment experiments, various concentrations of the test compounds were applied to one side of the mouse ear at specific times after the HD exposure. The antivesicant activities of the epicatechin compounds were measured by the percentage ear weight reduction of the treated ear versus the untreated control ear weight of the same mouse. The results are shown in Table 1 at Example 1.

The results indicated that the nucleophilicity of epicatechins and epicatechin oligomers relates to the efficacy of the compound as an antivesicant.

The protectant or decontaminant include clothing, combat gear, protective shelters, weapons, equipment, filters, sponges, foams, sprays, lotions, gases and the like which may be used to protect against or prevent injuries induced by vesicant agents or may be used to decontaminate persons or objects exposed to vesicant agents.

The terms and abbreviations used in the instant disclosure have their normal meanings unless otherwise designated. As used in the present application, the following definitions apply:

As used herein, “antivesicant induced injuries” include those caused by exposure to vesicant agents such as sulfur mustard (bis-2-chloroethylsulfide (HD)), Nitrogen mustard (Mustargen7), Lewisite, phosgene oxime, and combinations thereof. As used herein, “HD-induced injuries” are injuries caused by exposure to HD compounds and combinations comprising HD such as HD Lewisite (HL). Such antivesicant and HD induced injuries include damage to skin, eyes, lungs, including upper and lower airways, and systemic effects such as bone marrow suppression.

As used herein, “antivesicant activity” refers to the activity of a compound which prevents, inhibits or modulates an injury induced by a vesicant agent.

As used herein, “antivesicant” or “antivesicant compound” refers to a compound which exhibits antivesicant activity.

In accordance with a convention used in the art,

is used in structural formulas herein to depict the bond that is the point of attachment of the moiety or substituent to the core or backbone structure. Additionally,

used in the schematic rationale above is used to depict the bonds that are the point of attachment of either two substituents, which may or may not be the same, or a ring structure.

Where chiral carbons are included in chemical structures, unless a particular orientation is depicted, both sterioisomeric forms are intended to be encompassed.

It is understood that while a compound of the general structural formulas herein may exhibit the phenomenon of tautomerism, the structural formulas within this specification expressly depict only one of the possible tautomeric forms. It is therefore to be understood that the structural formulas herein are intended to represent any tautomeric form of the depicted compound and is not to be limited merely to a specific compound form depicted by the structural formulas.

It is also understood that the structural formulas are intended to represent any configurational form of the depicted compound and is not to be limited merely to a specific compound form depicted by the structural formulas.

Some of the antivesicants may exist as single stereoisomers (i.e., essentially free of other stereoisomers), racemates, or mixtures of enantiomers, diastereomers, or both. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, if the compounds of the present invention are made synthetically, they are used in a form that is at least 90% optically pure, that is, a form that comprises at least 90% of a single isomer (80% enantiomeric excess (e.e.) or diastereomeric excess (d.e.), more preferably at least 95% (90% e.e. or d.e.), even more preferably at least 97.5% (95% e.e. or d.e.), and most preferably at least 99% (98% e.e. or d.e.).

The antivesicant compounds of the present invention may be prepared using reaction routes, synthesis schemes and techniques available in the art using starting materials that are readily available. The antivesicant compounds of the present invention were made according to the following schemes and methods. However, it should be noted that the antivesicant compounds of the present invention may be made by other methods known in the art.

“A pharmaceutically acceptable prodrug” is a compound that may be converted under physiological conditions or by solvolysis to the specified compound or to a pharmaceutically acceptable salt of such compound. “A pharmaceutically active metabolite” is intended to mean a pharmacologically active product produced through metabolism in the body of a specified compound or salt thereof. Prodrugs and active metabolites of a compound may be identified using routine techniques known in the art. See, e.g., Bertolini, G. et al., (1997) J. Med. Chem. 40:2011-2016; Shan, D. et al., J. Pharm. Sci., 86(7):765-767; Bagshawe K., (1995) Drug Dev. Res. 34:220-230; Bodor, N., (1984) Advances in Drug Res. 13:224-331; Bundgaard, H., Design of Prodrugs (Elsevier Press, 1985); and Larsen, I. K., Design and Application of Prodrugs, Drug Design and Development (Krogsgaard-Larsen et al., eds., Harwood Academic Publishers, 1991).

If the antivesicant compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyrvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the antivesicant compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from basic amino acids, such as lysine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. Preferred amines and salts thereof are those that are clinically acceptable, i.e. not too toxic in the subject being treated.

In the case of compounds that are solids, it is understood by those skilled in the art that the inventive compounds and salts may exist in different crystal or polymorphic forms, all of which are intended to be within the scope of the present invention and specified structural formulas.

The antivesicant activity of the antivesicant compounds of the present invention may be measured by any of the methods available to those skilled in the art, including in vitro and in vivo assays. Examples of suitable assays for activity measurements are provided herein. Properties of the antivesicant compounds may be assessed, for example, by using one or more of the assays set out in the Examples below. Other pharmacological methods may also be used to determine the efficacy of the compounds as antivesicant compounds.

The antivesicant compounds in accordance with the present invention are useful in the treatment of antivesicant induced injuries, preferably HD-induced injuries and the like. Such antivesicant and HD induced injuries include cutaneous, ocular and pulmonary injuries such as damage to skin, eyes, lungs, including upper and lower airways, and systemic effects such as bone marrow suppression.

The antivesicant compounds of the present invention may be used in combination with or as a substitution for treatments of the above conditions. For example, the antivesicant compounds may also be used alone or in combination with a supplementary active compound such as anti-inflammatory and anti-protease drugs and the like to treat, prevent or inhibit antivesicant induced injuries such as HD-induced injuries associated with exposure to HD compounds and derivatives.

An antivesicant compound of the present invention may be administered in a therapeutically effective amount to a mammal such as a human. A therapeutically effective amount may be readily determined by standard methods known in the art. As used herein, a “therapeutically effective amount” of an antivesicant compound of the present invention is an amount which prevents, inhibits, suppresses or reduces the amount of injury or damage caused by exposure to a vesicant agent, such as an HD compound or derivative thereof, in a subject as compared to a control.

As defined herein, a therapeutically effective amount of a compound of the present invention may be readily determined by one of ordinary skill by routine methods known in the art. Preferred topical concentrations include about 0.1% to about 10% in a formulated salve. The skilled artisan will appreciate that certain factors may influence the dosage required to effectively treat a subject, including but not limited to the severity of the disease or disorder, previous treatments, the general health and/or age of the subject, and other diseases present.

Moreover, treatment of a subject with a therapeutically effective amount of the antivesicant compound preferably includes a single treatment, but can include a series of treatments. For example, a subject may be treated with an antivesicant compound of the invention at least once. However, the subject may treated with the antivesicant compound from about one time per week to about several times daily for a given treatment period. The length of the treatment period will depend on a variety of factors such as the length of exposure to the vesicant agent, the severity of the injury, the predisposition of exposure to a vesicant compound, or a combination thereof. It will also be appreciated that the effective dosage of the compound used for treatment may increase or decrease over the course of a particular treatment. Changes in dosage may result and become apparent by standard diagnostic assays known in the art. In some instances chronic administration may be required. The antivesicant compound may be administered before, during, after, or a combination thereof exposure to a vesicant agent.

The pharmaceutical compositions of the invention may be prepared in a unit-dosage form appropriate for the desired mode of administration. The compositions of the present invention may be administered for therapy by any suitable route including oral, rectal, nasal, topical (including buccal and sublingual), vaginal and parenteral (including subcutaneous, intramuscular, intravenous and intradermal). It will be appreciated that the preferred route will vary with the condition and age of the recipient, the nature of the condition to be treated, and the chosen active compound.

It will be appreciated that the actual dosages of the compounds used in the compositions of this invention will vary according to the particular complex being used, the particular composition formulated, the mode of administration, and the particular site, host, and disease being treated. Optimal dosages for a given set of conditions may be ascertained by those skilled in the art using conventional dosage-determination tests in view of the experimental data for a given antivesicant compound. Administration of prodrugs may be dosed at weight levels that are chemically equivalent to the weight levels of the fully active forms.

The antivesicant compounds of the invention can be incorporated into pharmaceutical compositions suitable for administration. Pharmaceutical compositions of this invention comprise a therapeutically effective amount of an antivesicant compound having the Structural Formula A, and an inert, pharmaceutically acceptable carrier or diluent. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like. The use of such media and agents for pharmaceutically active substances is well known in the art.

Except insofar as any conventional media or agent is incompatible with the active compound, use thereof in the compositions is contemplated. Supplementary active compounds can also be incorporated into the compositions. Supplementary active compounds include anti-inflammatory and anti-protease drugs and other compounds commonly used to treat injuries induced by exposure to vesicant agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent is dissolved in an aqueous solution of an organic or inorganic acid, such as 0.3M solution of succinic acid or citric acid. If a soluble salt form is not available, the agent may be dissolved in a suitable co-solvent or combinations of co-solvents. Examples of suitable co-solvents include, but are not limited to, alcohol, propylene glycol, polyethylene glycol 300, polysorbate 80, glycerin and the like in concentrations ranging from 0-60% of the total volume. In an exemplary embodiment, the antivesicant compound of the present invention is dissolved in DMSO and diluted with water.

The composition may also be in the form of a solution of a salt form of the active ingredient in an appropriate aqueous vehicle such as water or isotonic saline or dextrose solution.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (compound), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally comprise gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds and agents.

Pharmaceutical preparations which can be used orally include push-fit capsules made of gelatin, as well as soft, sealed capsules made of gelatin and a plasticizer, such as glycerol or sorbitol. The push-fit capsules can comprise the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can comprise any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Preferred formulations for oral formulations include microcrystalline tablets, gelatin capsules, or the like.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated comprising a powder mix of the compound and a suitable powder base such as lactose or starch. Nebulizers, which will deliver a finite proportion of particles into the deep lung, as opposed to undesired deposition in the upper bronchial tree. are well known in the prior art.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may comprise formulatory agents such as suspending, stabilizing and/or dispersing agents.

Pharmaceutical compositions suitable for injectable use include sterile aqueous solutions (where water soluble) or dispersions and sterile powders for the extemporaneous preparation of sterile injectable solutions or dispersion. Aqueous injection suspensions may comprise substances which increase the viscosity of the suspension, such as sodium carboxymethyl cellulose, sorbitol, or dextran. Optionally, the suspension may also comprise suitable stabilizers or agents which increase the solubility of the compounds to allow for the preparation of highly concentrated solutions. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium comprising, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a therapeutically effective amount of a compound of the invention in an appropriate solvent with one or a combination of ingredients enumerated above, as required, followed by filtered sterilization. Generally, dispersions are prepared by incorporating the antivesicant compound into a sterile vehicle which comprises a basic dispersion medium and the required other ingredients from those enumerated above. In the case of sterile powders for the preparation of sterile injectable solutions, the preferred methods of preparation are vacuum drying and freeze-drying which yields a powder of the active compound plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, foams, powders, sprays, aerosols or creams as generally known in the art.

For example, for topical formulations, pharmaceutically acceptable excipients may comprise solvents, emollients, humectants, preservatives, emulsifiers, and pH agents. Suitable solvents include ethanol, acetone, glycols, polyurethanes, and others known in the art. Suitable emollients include petrolatum, mineral oil, propylene glycol dicaprylate, lower fatty acid esters, lower alkyl ethers of propylene glycol, cetyl alcohol, cetostearyl alcohol, stearyl alcohol, stearic acid, wax, and others known in the art. Suitable humectants include glycerin, sorbitol, and others known in the art. Suitable emulsifiers include glyceryl monostearate, glyceryl monoleate, stearic acid, polyoxyethylene cetyl ether, polyoxyethylene cetostearyl ether, polyoxyethylene stearyl ether, polyethylene glycol stearate, propylene glycol stearate, and others known in the art. Suitable pH agents include hydrochloric acid, phosphoric acid, diethanolamine, triethanolamine, sodium hydroxide, monobasic sodium phosphate, dibasic sodium phosphate, and others known in the art. Suitable preservatives include benzyl alcohol, sodium benzoate, parabens, and others known in the art.

For administration to the eye, the compound of the invention is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be an ointment, vegetable oil, or an encapsulating material. A compound of the invention may also be injected directly into the vitreous and aqueous humor.

Alternatively, the active ingredient may be in powder form for constitution with a suitable vehicle, e.g., sterile pyrogen-free water, before use. The compounds may also be formulated in rectal compositions such as suppositories or retention enemas, e.g., comprising conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) comprises VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied, for example: other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers comprising the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit comprising a predetermined quantity of active compound calculated to produce the desired therapeutic effect in association with the required pharmaceutical carrier.

The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pulmonary Damage

1. Inflammatory Processes in Asthma and Chronic Obstructive Pulmonary Disease

Mammalian inflammatory pathways are an important consequence of the immune system and play a vital role in the normal homeostasis of the body. Whilst short-term inflammation has a protective function, in chronic diseases such as arthritis and asthma, inflammation is associated with the typical oedema, swelling, pain and organ dysfunction. Prostaglandins and leukotrienes are potent biologically active structures that normally play an essential role in tissue homeostasis. However, following cellular injury or trauma the respective production of specific prostaglandins and leukotrienes shifts to an inflammatory reaction with local physiological effects

Eicosanoid Metabolism

Eicosanoids are 20-carbon compounds derived from polyunsaturated fatty acids, also known as the eicosanoic acids and which serve as precursors to a variety of other biologically active compounds within cells. These include prostaglandins, thromboxanes and leukotrienes, which are themselves eicosanoids and are therefore based upon the eicosanoid 20-carbon structure.

At the cellular level, arachidonic acid is one of the major sources of 20-carbon structures which provide the essential precursors of prostaglandins (sometimes referred to as Prostanoids), thromboxanes and leukotrienes. These compounds act as biological regulators within animals and their function depends upon the type of tissue and relevant enzyme systems involved and are well known mediators of inflammation and immune response.

Eicosanoid metabolism is controlled by the availability of arachidonic acid or other eicosanoid structures, enzyme expression and negative or positive feedback loops for example. Eicosanoids are potent regulators of cell metabolism but have a short half-life of less than 5 minutes allowing for significant control over physiological functions. Their potency is such that the ratio of body mass to eicosanoid mass is in the order of 1 million.

In recent years pharmacological research has begun to unravel the complexities of mammalian inflammatory pathways leading to increased pharmaceutical interest in novel compounds that can provide anti-inflammatory activity with reduced adverse effects, contra-indications or toxicity.

### Eicosanoids and the Inflammatory Process

The inflammatory process begins with cell injury. Trauma, infection, or other injury to the cell which activates membrane bound phospholipase A2 (pLA2), which releases arachidonic acid from the injured cell's membrane. Arachidonic acid fuels the cyclo-oxygenase and lipoxygenase inflammatory pathways.

The inflammatory process directly involves eicosanoid metabolism. Of the numerous mechanisms involved a number of pathways are of particular interest, the cyclo-oxygenase (or COX) and lipoxygenase (LOX) pathways, both of which constitute the Arachidonic Acid Cascade.

The arachidonic acid cascade is responsible for the production of various biological regulators at the tissue level. Control of eicosanoid metabolism can be achieved by the supply of arachidonic acid, negative feedback mechanisms and therapeutically by treatment with non-steroidal anti-inflammatory drugs (NSAIDs) for example. The biochemical by-products of this process have been implicated in many divergent physiologic responses to inflammation.

### The Lipoxygenase Pathway

Lipoxygenase is an enzyme that converts arachidonic acid to several intermediates, including 5-hydroperoxyeicosatetraenoic acid (5-HPETE), which gives rise to the leukotrienes (LTA4, LTB4, LTC4, and LTD4). Leukotrienes play a role in vascular permeability and they are potent chemotactic factors, increasing White Blood Cell (WBC) migration into inflamed tissues. Leukotrienes are associated with the development of oedema and WBC effusion into tissues such as joints and lung endothelium in arthritis and asthma respectively. Recently a number of anti-leukotriene therapies have been licensed for the treatment of asthma Most research has concentrated on NSAIDs demonstrating varying efficacies. The widely varying profiles of currently available NSAIDs may be explained by the discovery of two isoforms of the cyclo-oxygenase enzyme possessing different profiles. Cyclooxygenase 1 (COX 1) has a physiological role and influences the normal activities of platelet aggregation, gastric mucosa, and kidney. COX1 activity is not influenced by inflammatory stimulation. Cyclooxygenase 2 (COX 2) is induced by inflammatory stimulation releasing pro-inflammatory prostaglandins.

### The Use of Epicatechins and Epicatechin Oligomers in the Management of Asthma and Chronic Obstructive Airway Disease

Flavonoids constitute an important group of dietary polyphenols, which are widely distributed in plants. Over 4000 different flavonoids have been described, and they are categorized into flavonols, flavones, flavanones, anthocyanidins, and isoflavones. In general, particularly rich dietary sources of flavonoids are red grape juice, red wine, green and black tea, cocoa and chocolate, various fruits, green vegetables and onions. Some of the flavonols occur as covalently linked oligomers, the procyanidins. Although the flavonoids do not belong to the vitamins, their daily intake is in the same order of magnitude of that of the antioxidant vitamins C and E. Therefore they are classified as micronutrients. The flavonoids and other dietary polyphenols contribute to the antioxidant defence system of the organism against oxidative stress. Flavonoids have also been reported to exert anticancer and antimicrobial activities. A number of in vitro and in vivo studies as well as clinical trials suggest beneficial effects of flavonoids for health. In particular, high intake of flavonoids is believed to counteract the development of cardiovascular diseases. Flavonoids have demonstrated a variety of biological effects including anti-oxidation, anti-inflammation, anti-allergic effects, anti-platelet, and anti-thrombotic actions.

For example, an in vitro oxidation model showed quercetin, myricetin, and rutin are more efficient antioxidants than traditional vitamins. Some flavonoids, especially quercetin, protect low-density lipoprotein from oxidative damage in vitro and are thought capable of reducing the risk of coronary heart disease or cancer. Flavonols and flavones also have antioxidant and free radical scavenging activity in foods. Epidemiological studies have indicated a relationship between a diet rich in flavonols and a reduced incidence of heart disease. Others, such as the anthocyanidins from some purple plant foods may help protect the lens of the eye. Soy isoflavones are also currently being studied to see if they help fight cancer. Quercetin has been reported to block the “sorbitol pathway” which is linked to many problems associated with diabetes. Rutin and several other flavonoids may also protect blood vessels. Their mode of antioxidant action appears to be multivalent and occurs at three different levels: (i) scavenging of free radicals and reactive oxygen and nitrogen species, (ii) chelating of transition metal ions, thus masking the pro-oxidant actions, (iii) ameliorating deleterious actions of pro-oxidant enzymes (lipoxygenases, myeloperoxidase and others). The USDA Database for the Flavonoid Content of Selected Foods, released in March 2003, contains information on the most prevalent dietary flavonoids. These are organized into five subclasses based on their chemical structure:

### Flavonols

Quercetin, Rutin (a glycosylated form of quercetin), Kaempferol, Myricetin, Isorhamnetin,

### Flavones

Apigenin, Luteolin

### Flavanones

Hesperetin, Naringenin, Eriodictyol

### Flavan-3-Ols

Catechins, Epicatechins, Theaflavins, Thearubigins

### Anthocyanidins

Cyanidin, Delphinidin, Malvidin, Pelargonidin, Peonidin, Petunidin

The flavonoids are components of many common vegetables. For instance, the flavones (the group containing luteolin) are found in celery green hearts, celery, parsley and rutabagas and other sources.

We are principally concerned here with epicatechins and epicatechin oligomers. The present invention makes use of the combined anti-inflammatory properties and antioxidant properties of epicatechins and epicatechin oligomers. More specifically the instant invention utilizes the properties of mixtures of epicatechins and epicatechin oligomers in the form that they are present as natural products as extracted from plant sources rather than as highly purified synthetic compounds, known to those normally skilled in the art as natural products. Even more specifically, the said epicatechins and epicatechin oligomers are in the form in which they are obtained by extraction from seeds of the cacao tree Theobroma cacao. The synergistic effect of combined cacao epicatechins and epicatechin oligomers upon asthma, chronic obstructive pulmonary disease and rheumatoid and osteoarthritis, and other inflammatory conditions.

Pulmonary Administration of Epicatechins and Epicatechin Oligomers

The active substance according to the invention are preferably administered by pulmonary inhalation. For this purpose, cacao extract containing epicatechin and epicatechin oligomers has to be made available in inhalable forms. Inhalable preparations include, in particular, inhalable powders. Inhalable powders according to the invention containing the cacao extract containing epicatechin and epicatechin oligomers consist of the active substances on their own or of a mixture of the active substances with physiologically acceptable excipients. The preparations according to the invention may contain the, cacao extract containing epicatechin and epicatechin oligomers either together in one formulation or in two separate formulations. These formulations which may be used within the scope of the present invention are described in more detail in the next part of the specification.

The pharmaceutical compositions according to the invention are usually administered by giving cacao extract containing epicatechin and epicatechin oligomers in doses from 0.01 to 10000 micrograms/g, preferably from 0.1 to 2000 micrograms/g, more preferably from 10 to 1000 micrograms/g, still more preferably from 15 to 500 micrograms/g per single dose.

For example, compositions according to the invention contain an amount of cacao extract containing epicatechin and epicatechin oligomers such that the total dose per single dose in the ratio by weight of 15 micrograms/g, 20 micrograms/g, 25 micrograms/g, 30 micrograms/g, 35 micrograms/g, 45 micrograms/g, 50 micrograms/g, 55 micrograms/g, 60 micrograms/g, 65 micrograms/g, 70 micrograms/g 75 micrograms/g, 80 micrograms/g, 85 micrograms/g, 90 micrograms/g, 95 micrograms/g, 100 micrograms/g, 105 micrograms/g, 110 micrograms/g, 115 micrograms/g, 120 micrograms/g, 125 micrograms/g, 130 micrograms/g, 135 micrograms/g, 140 micrograms/g, 145 micrograms/g, 150 micrograms/g, 155 micrograms/g, 160 micrograms/g, 165 micrograms/g, 170 micrograms/g, 175 micrograms/g, 180 micrograms/g, 185 micrograms/g, 190 micrograms/g, 195 micrograms/g, 200 micrograms/g, 205 micrograms/g, 210 micrograms/g, 215 micrograms/g, 220 micrograms/g, 225 micrograms/g, 230 micrograms/g, 235 micrograms/g, 240 micrograms/g, 245 micrograms/g, 250 micrograms/g, 255 micrograms/g, 260 micrograms/g, 265 micrograms/g, 270 micrograms/g, 275 micrograms/g, 280 micrograms/g, 285 micrograms/g, 290 micrograms/g, 295 micrograms/g, 300 micrograms/g, 305 micrograms/g, 310 micrograms/g, 315 micrograms/g, 320 micrograms/g, 325 micrograms/g, 330 micrograms/g, 335 micrograms/g, 340 micrograms/g, 345 micrograms/g, 350 micrograms/g, 355 micrograms/g, 360 micrograms/g, 365 micrograms/g, 370 micrograms/g, 375 micrograms/g, 380 micrograms/g, 385 micrograms/g, 390 micrograms/g, 395 micrograms/g, 400 micrograms/g, 405 micrograms/g, 410 micrograms/g, 415 micrograms/g, 420 micrograms/g, 425 micrograms/g, 430 micrograms/g, 435 micrograms/g, 440 micrograms/g, 445 micrograms/g, 450 micrograms/g, 455 micrograms/g, 460 micrograms/g, 465 micrograms/g, 470 micrograms/g, 475 micrograms/g, 480 micrograms/g, 485 micrograms/g, 490 micrograms/g, 495 micrograms/g, 500 micrograms/g, 505 micrograms/g, or the like.

In some embodiments in which placement of the pharmaceutical compositions in the deep lung is desirable, particles can be between about 0.2 to about 0.8 micrometer. The dose can be between about 10-20 mg and about 100 mg. In some embodiments, the method of administration can be with either a dry powder inhaler or a fluid nebulizer. An exemplary dose for COPD or asthma can be in excess of 100-200 mg over a 30-minute session.

### A) Inhalable Powder Containing the Cacao Extract Containing Epicatechin and Epicatechin Oligomers According to the Invention:

The inhalable powders according to the invention may contain cacao extract containing epicatechin and epicatechin oligomers either on their own or in admixture with suitable physiologically acceptable excipients.

If the cacao extract containing epicatechin and epicatechin oligomers are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrane), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred. Within the scope of the inhalable powders according to the invention the excipients have a maximum average particle size of up to 250 micrometers, preferably between 10 and 150 micrometers, most preferably between 15 and 80 micrometers. It may sometimes seem appropriate to add finer excipient fractions with an average particle size of 1 to 9 micrometers to the excipients mentioned above. These finer excipients are also selected from the group of possible excipients listed hereinbefore. In particularly preferred inhalable powders the excipient is characterised by an average particle size of 12 to 35 micrometers, more preferably from 13 to 30 micrometers. Also particularly preferred are inhalable powders in which the 10% fine content is about 1 to 4 micrometers, preferably about 1.5 to 3 micrometers.

/By the average particle size is meant here the 50% value of the volume distribution measured using a laser diffractometer (Malvern Instruments Inc.) by the dry dispersion method. Analogously, the 10% fine content in this instance refers to the 10% value of the volume distribution measured using a laser diffractometer.

Preferably, excipients of high crystallinity are used for the powder formulations according to the invention. This crystallinity can be assessed by means of the enthalpy released as the excipient is dissolved (solution enthalpy). In the case of the excipient lactose monohydrate, which is most preferably used according to the invention, it is preferable to use lactose which is characterised by a high solution enthalpy.

Finally, in order to prepare the inhalable powders according to the invention, micronised active substance preferably with an average particle size of 0.5 to 10 micrometers, more preferably from 1 to 5 micrometers, are added to the excipient mixture.

In the case of the active cacao extract containing epicatechin and epicatechin oligomers according to the invention the following procedure has proved particularly suitable for micronising this crystalline active substance modification. The process may be carried out using conventional mills. Preferably, the micronisation is carried out with the exclusion of moisture, more preferably, using a corresponding inert gas such as nitrogen, for example. It has proved particularly preferable to use air jet mills in which the material is comminuted by the impact of the particles on one another and on the walls of the grinding container.

According to the invention, nitrogen is preferably used as the grinding gas. The material for grinding is conveyed by the grinding gas under specific pressures (grinding pressure). Within the scope of the present invention, the grinding pressure is usually set to a value between about 2 and 8 bar, preferably between about 3 and 7 bar, most preferably between about 3.5 and 6.5 bar. The material for grinding is fed into the air jet mill by means of the feed gas under specific pressures (feed pressure). Within the scope of the present invention a feed pressure of between about 2 and 8 bar, preferably between about 3 and 7 bar and most preferably between about 3.5 and 6 bar has proved satisfactory. The feed gas used is also preferably an inert gas, most preferably nitrogen again. The material to be ground (crystalline cacao extract containing epicatechin and epicatechin oligomers) may be fed in at a rate of about 5-35 g/min, preferably at about 10-30 g/min.

For example, without restricting the subject of the invention thereto, the following apparatus has proved suitable as a possible embodiment of an air jet mill: a 2-inch Microniser with grinding ring, 0.8 mm bore, made by Sturtevant Inc., 348 Circuit Street, Hanover, Mass. 02239, USA. Using this apparatus, the grinding process is preferably carried out with the following grinding parameters: grinding pressure: about 4.5-6.5 bar; feed pressure: about 4.5-6.5 bar; supply of grinding material: about 17-21 g/min.

The ground material thus obtained is then further processed under the following specific conditions. The micronisate is exposed to water vapour at a relative humidity of at least 40% at a temperature of 15-40 deg/C., preferably 20-35 deg/C., most preferably 25-30 deg/C. Preferably, the humidity is set to a value of 50-95% r.h., preferably 60-90% r.h., most preferably 70-80% r.h. By relative humidity (r.h.) is meant the quotient of the partial steam pressure and the steam pressure of the water at the temperature in question. Preferably, the micronisate obtained from the grinding process described above is subjected to the chamber conditions mentioned above for a period of at least 6 hours. Preferably, however, the micronisate is subjected to the chamber conditions mentioned above for about 12 to 48 hours, preferably about 18 to 36 hours, more preferably about 20 to 28 hours.

The micronisate of cacao extract containing epicatechin and epicatechin oligomers obtainable by the above method has a characteristic particle size of between 1.0 micrometers and 3.5 micrometers, preferably between 1.1 micrometers and 3.3 micrometers, most preferably between 1.2 micrometers and 3.0 micrometers and Q(5.8) of more than 60%, preferably more than 70%, most preferably more than 80%. The characteristic value Q(5.8) indicates the quantity of particles below 5.8 micrometers, based on the volume distribution of the particles. The particle sizes were determined within the scope of the present invention by laser diffraction.

Also characteristic of the cacao extract containing epicatechin and epicatechin oligomers micronisate according to the invention which was prepared by the above process are Specific Surface Area values in the range between 2 square meters/g and 5 square meters/g, more particularly between 2.5 square meters/g and 4.5 square meters/g and most outstandingly between 3.0 square meters/g and 4.0 square meters/g.

The inhalable powders according to the invention may be administered using various types of inhalers known from the prior art.

Inhalable powders according to the invention which contain a physiologically acceptable excipient in epicatechin and epicatechin oligomers may be administered, for example, by means of inhalers which deliver a single dose from a supply using a measuring chamber as described in U.S. Pat. No. 4,570,630A, or by other means as described in DE 36 25 685 A. Preferably, the inhalable powders according to the invention which contain physiologically acceptable excipient in addition to cacao extract containing epicatechin and epicatechin oligomers are packed into capsules (to produce so-called inhalettes) which are used in inhalers as described, for example, in WO 94/28958.

A particularly preferred inhaler for administering the pharmaceutical combination according to the invention is a spinhaler.

For administering the inhalable powders according to the invention with a spinhaler using powder-filled capsules it is particularly preferred to use capsules the material of which is selected from among the synthetic plastics, most preferably selected from among polyethylene, polycarbonate, polyester, polypropylene and polyethylene terephthalate. Particularly preferred synthetic plastic materials are polyethylene, polycarbonate or polyethylene terephthalate. If polyethylene is used as one of the capsule materials which is particularly preferred according to the invention, it is preferable to use polyethylene with a density of between 900 and 1000 kg/m3, preferably 940-980 kg/m3, more preferably about 960-970 kg/m3 (high density polyethylene).

The synthetic plastics according to the invention may be processed in various ways using manufacturing methods known in the art. Injection moulding of the plastics is preferred according to the invention. Injection moulding without the use of mould release agents is particularly preferred. This method of production is well defined and is characterised by being particularly reproducible.

In another aspect the present invention relates to the abovementioned capsules which contain the abovementioned inhalable powders according to the invention. If the inhalable powders according to the invention are intended to be packed into capsules (inhalettes) for the preferred use described above, fill amounts of from 1 to 30 mg, preferably from 3 to 20 mg, preferably 5 to 10 mg of inhalable powder per capsule are recommended. These contain, according to the invention, either together or separately, the abovementioned dosages of cacao extract containing epicatechin and epicatechin oligomers per single dose. As already mentioned, the present invention also relates to a kit consisting of two capsules each of which contains the active substances cacao extract containing epicatechin and epicatechin oligomers optionally combined with one of the abovementioned physiologically acceptable excipients.

The present invention also relates to an inhalation kit consisting of one or more of the above capsules characterised by a content of inhalable powder consisting of cacao extract containing epicatechin and epicatechin oligomers according to the invention in conjunction with a spinhaler

The present invention also relates to the use of the abovementioned capsules characterised by a content of inhalable powder consisting of cacao extract containing epicatechin and epicatechin oligomers according to the invention, for preparing a pharmaceutical composition for treating respiratory complaints, especially for treating COPD and/or asthma.

Filled capsules which contain the inhalable powders according to the invention are produced by methods known in the art, by filling the empty capsules with the inhalable powders according to the invention.

### B) Propellant Gas-Driven Inhalation Aerosols Consisting of Cacao Extract Containing Epicatechin and Epicatechin Oligomers

Inhalation aerosols containing propellant gas according to the invention may contain substances cacao extract containing epicatechin and epicatechin oligomers dissolved in the propellant gas or in dispersed form. cacao extract containing epicatechin and epicatechin oligomers may be present in separate formulations or in a single preparation, in which cacao extract containing epicatechin and epicatechin oligomers are either each dissolved, dispersed or only one or two of the components is or are dissolved and the other or others is or are dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG134a, TG227 and mixtures thereof.

The propellant-driven inhalation aerosols according to the invention may also contain other ingredients such as co-solvents, stabilisers, surfactants, antioxidants, lubricants and pH adjusters. All these ingredients are well known to those normally skilled in the art.

The inhalation aerosols containing propellant gas according to the invention may contain up to 5 wt.-% of active substance Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active cacao extract containing epicatechin and epicatechin oligomers.

If the cacao extract containing epicatechin and epicatechin oligomers are present in dispersed form, the particles of active substance preferably have an average particle size of up to 10 micrometers, preferably from 0.1 to 5 micrometers, more preferably from 1 to 5 micrometers. This may optionally be used in the form of the micronisate described in more detail in the previous section.

The propellant-driven inhalation aerosols according to the invention mentioned above may be administered using inhalers known in the art (MDIs=metered dose inhalers).

Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

### C) Propellant-Free Inhalable Solutions or Suspensions Consisting of the Cacao Extract Containing Epicatechin and Epicatechin Oligomers According to the Invention:

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing cacao extract containing epicatechin and epicatechin oligomers, separately or together, are adjusted to a pH of 2 to 7, preferably 2 to 5, using suitable acids.

The pH may be adjusted using acids selected from inorganic or organic acids. Examples of suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example.

According to the invention, it is particularly preferred to use hydrochloric acid to adjust the pH.

According to the invention, the addition of EDTA or one of the known salts thereof, as stabiliser or complexing agent is unnecessary in the present formulation. Other embodiments may contain this compound or these compounds. In a preferred embodiment the content based on disodium EDTA is less than 100 mg/100 ml, preferably less than 50 mg/100 ml, more preferably less than 20 mg/100 ml. Generally, inhalable solutions in which the content of disodium EDTA is from 0 to 10 mg/100 ml are preferred.

According to the invention, the addition of ascorbic acid or one of the known salts thereof, as stabiliser or antioxidant is unnecessary in the present formulation. Other embodiments may contain this compound or these compounds. In a preferred embodiment the content based on ascorbic acid is less than 100 mg/100 ml, preferably less than 50 mg/100 ml, more preferably less than 20 mg/100 ml. Generally, inhalable solutions in which the content of ascorbic acid is from 0 to 10 mg/100 ml are preferred.

According to the invention, the addition of palmitate ester of ascorbic acid or one of the known salts thereof, as stabiliser or antioxidant is unnecessary in the present formulation. Other embodiments may contain this compound or these compounds. In a preferred embodiment the content based on palmitate ester of ascorbic acid is less than 100 mg/100 ml, preferably less than 50 mg/100 ml, more preferably less than 20 mg/100 ml. Generally, inhalable solutions in which the content of palmitate ester of ascorbic acid is from 0 to 10 mg/100 ml are preferred.

According to the invention, the addition of tocopherols or one of the known salts thereof, as stabiliser or antioxidant is unnecessary in the present formulation. Other embodiments may contain this compound or these compounds. In a preferred embodiment the content based on tocopherols is less than 100 mg/100 ml, preferably less than 50 mg/100 ml, more preferably less than 20 mg/100 ml. Generally, inhalable solutions in which the content of tocopherols is from 0 to 10 mg/100 ml are preferred.

According to the invention, the addition of lipoic acid or one of the known salts thereof, as stabiliser or antioxidant is unnecessary in the present formulation. Other embodiments may contain this compound or these compounds. In a preferred embodiment the content based on lipoic acid is less than 100 mg/100 ml, preferably less than 50 mg/100 ml, more preferably less than 20 mg/100 ml. Generally, inhalable solutions in which the content of lipoic acid is from 0 to 10 mg/100 ml are preferred. The lipoic acid may be in the racemic form or present as the (R) or (S) enantiomer.

Cosolvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin (asolectin), cocoa butter, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include physiologically acceptable salts such as sodium chloride as isotonic agents.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

/Preferred formulations contain, in addition to the solvent water and the combination of active substances cacao extract containing epicatechin and epicatechin oligomers, only benzalkonium chloride and disodium EDTA. In another preferred embodiment, no disodium EDTA is present.

The propellant-free inhalable solutions according to the invention are administered in particular using inhalers of the kind which are capable of nebulising a small amount of a liquid formulation in the required therapeutic dose within a few seconds to produce an aerosol suitable for therapeutic inhalation. Within the scope of the present invention, preferred nebulisers are those in which a quantity of less than 100 microliters, preferably less than 50 microliters, more preferably between 20 and 30 microliters of active substance solution can be nebulised in preferably one spray action to form an aerosol with an average particle size of less than 20 micrometers, preferably less than 10 micrometers, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687.

The propellant-free inhalable solutions or suspensions according to the invention may take the form of concentrates or sterile inhalable solutions or suspensions ready for use, as well as the above-mentioned solutions and suspensions. Sterile formulations ready for use may be administered using energy-operated fixed or portable nebulisers which produce inhalable aerosols by means of ultrasound or compressed air by the Venturi principle or other principles.

### Pulmonary Injury Induced by Smoke Inhalation

The United States has one of the world's largest per capita fire death rates. House fires alone kill more than 9,000 Americans annually, and smoke inhalation is the leading cause of mortality from structural fires. Smoke inhalation injury is a serious threat to victims of house fires, explosions, and other disasters involving fire and smoke. This type of injury alone can be lethal as shown in the Cocoanut Grove fire, in which 492 people died, most without burns. In the Rhode Island nightclub fire, 95 people died (out of 350 victims and survivors of this tragedy), and 187 people were treated for smoke inhalation lung injury and burns.

Inhalation injury results from the airway inflammatory response to inhalation of the products of incomplete combustion and is the leading cause of death (up to 77%) in burn patients. Approximately 33% of patients with extensive burns present inhalation injury, and the risk increases in proportion to the quantity of body surface area burned.

Smoke inhalation leads to the formation of obstructive airway debris, which may cause respiratory distress and increased mortality in victims of fire. Airway obstruction and smoke-induced lung apoptosis have been shown to occur in animal models with burn and smoke inhalation injuries but the mechanism of the smoke-induced airway damage has remained unclear.

The high mortality brought about by inhalation injuries is found in burns of all sizes but is most significant in burns which exceed 15% of the total body surface area. If severe enough, the pulmonary inflammatory response may promote Acute Respiratory Distress Syndrome (ARDS).

The clinical definition of ARDS is rapidly progressive bilateral alveolar infiltrates with Pao₂/fraction of inspired oxygen (Fio₂) ratio<200 and a pulmonary capillary wedge pressure<18 torr, or echocardiographic evidence of normal left ventricular function (American-European Consensus Conference). Pathologically, endothelial and epithelial injury with increased vascular permeability, interstitial pneumonitis, and extensive obliterative fibrosis with destruction of the normal lung architecture characterizes ARDS. Overall, ARDS affects 150,000 patients a year in the United States and results in approximately 30% mortality. ARDS associated with smoke inhalation and cutaneous burn (see above) is a predominant cause of death in burn patients. In a large retrospective study, the development of ARDS was found to be predictive of mortality, independent of the patient's age or size of the burn.

Injury related to the high temperature of the inhaled smoke rarely occurs in areas below the larynx. Multiple agents are probably responsible for the injury. First, oxygen concentration in a fire can be very low, leading to asphyxiation.

Most importantly of all is that combustion of synthetic materials, especially plastics, can lead to the formation of quite high concentrations of toxic gases. Among the various smoke components, acrolein, formaldehyde, sulfur dioxide and nitrogen dioxide can cause direct airway injury. Such injury results from an acute inflammatory process, mediated by polymorphonuclear leukocytes, especially neutrophils. Symptoms related to this process may not appear until 24 hours after exposure and may include changes in capillary permeability, lymphatic flow or mucociliary clearance, as well as acute respiratory distress syndrome or secondary infections. Other gases produced in combustion can produce systemic toxicity. Two gases are of particular importance: carbon monoxide and cyanide (both associated with high rates of morbidity and mortality). ARDS results as a consequence of a systemic inflammatory response. Apoptosis, programmed cell death, is frequently increased during injury and inflammation in parenchymal tissues. However, little is known about apoptotic processes in the lung after burn injury. This self-destructive process is regulated by both external and internal signals. Proapoptotic signals may be transduced through cytokine receptors of the tumor necrosis factor (TNF) family, eicosanoids, reactive oxygen species, proteases, and mechanical stretch. This signaling results in the cleavage and activation of cell-death proteases called caspases (cysteinyl aspartate-specific proteinases); of these, caspase-3 is commonly recognized as the initiator of apoptosis. This results in several changes within the cell leading to chromatin condensation and enzymatic fragmentation of DNA and ultimately death.

ARDS is treated primarily with mechanical ventilation, which tends to normalize blood gases through manipulation of minute ventilation and oxygen concentration, often allowing the lungs to recover from the inciting incident. However, mechanical ventilation may cause hyperinflation with alveolar stretch that may aggravate the pulmonary inflammatory response, dramatically altering the mechanical properties of the lungs and resulting in reduction of static compliance and inadequate gas exchange. Alternative gas exchange strategies in burned patients focus on reducing this mechanical ventilator-induced lung injury, and include low tidal volume (Vt), high-frequency percussive ventilation, and extracorporeal gas exchange techniques.

Aside from ARDS, Carbon monoxide intoxication is one of the most frequent causes of death in patients suffering from inhalation injury. Carbon monoxide has a high affinity for hemoglobin, which can be from 200 to 150 times higher than oxygen affinity with hemoglobin. The production of carboxyhemoglobin, an extremely stable complex, causes not only a decrease in oxyhemoglobin saturation, but also a shift of the dissociation curve to the left, reducing oxygen release to the tissues. In addition, competitive inhibition with cytochrome oxidase systems, especially cytochrome P450, impedes the use of oxygen for energy production. Carbon monoxide also binds to myoglobin, impairing oxygen storage in muscles.

Cyanide toxicity is caused by the inhibition of cellular oxygenation, causing tissue anoxia through reversible inhibition of cytochrome oxidase enzymes (Fe³⁺). The inhibition of the aerobic glycolytic pathway forces the metabolism onto the alternative anaerobic pathway, causing an accumulation of acid byproducts.

Particularly in the case of long-term injury from carbon monoxide free radical mediated reactive oxygen species (ROS) play an important role. The activation of the JNK pathway has been suggested to be important in the pathology of ARDS and more generally smoke-induced pulmonary damage of lesser severity which does not necessarily lead to ARDS.

There exists a serious need for agents which can be applied immediately after smoke exposure to ameliorate the multiple processes which result in smoke induced pulmonary damage and ARDS.

The present invention also relates to the use of the abovementioned capsules characterised by a content of inhalable powder consisting of cacao extract containing epicatechin and epicatechin oligomers according to the invention, for preparing a pharmaceutical composition for treating respiratory complaints, especially for treating SMOKE-INDUCED PULMONARY ACUTE RESPIRATORY DISTRESS and/or smoke-induced pulmonary acute respiratory distress.

Filled capsules which contain the inhalable powders according to the invention are produced by methods known in the art, by filling the empty capsules with the inhalable powders according to the invention.

### B) Propellant Gas-Driven Inhalation Aerosols Consisting of Cacao Extract Containing Epicatechin and Epicatechin Oligomers

Inhalation aerosols containing propellant gas according to the invention may contain substances cacao extract containing epicatechin and epicatechin oligomers dissolved in the propellant gas or in dispersed form. cacao extract containing epicatechin and epicatechin oligomers may be present in separate formulations or in a single preparation, in which cacao extract containing epicatechin and epicatechin oligomers are either each dissolved, dispersed or only one or two of the components is or are dissolved and the other or others is or are dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG134a, TG227 and mixtures thereof.

The propellant-driven inhalation aerosols according to the invention may also contain other ingredients such as co-solvents, stabilisers, surfactants, antioxidants, lubricants and pH adjusters. All these ingredients are well known to those normally skilled in the art.

The inhalation aerosols containing propellant gas according to the invention may contain up to 5 wt.-% of active substance Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active cacao extract containing epicatechin and epicatechin oligomers.

If the cacao extract containing epicatechin and epicatechin oligomers are present in dispersed form, the particles of active substance preferably have an average particle size of up to 10 micrometers, preferably from 0.1 to 5 micrometers, more preferably from 1 to 5 micrometers. This may optionally be used in the form of the micronisate described in more detail in the previous section.

The propellant-driven inhalation aerosols according to the invention mentioned above may be administered using inhalers known in the art (MDIs=metered dose inhalers).

Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

### C) Propellant-Free Inhalable Solutions or Suspensions Consisting of the Cacao Extract Containing Epicatechin and Epicatechin Oligomers According to the Invention:

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing cacao extract containing epicatechin and epicatechin oligomers, separately or together, are adjusted to a pH of 2 to 7, preferably 2 to 5, using suitable acids.

The pH may be adjusted using acids selected from inorganic or organic acids. Examples of suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example.

According to the invention, it is particularly preferred to use hydrochloric acid to adjust the pH.

According to the invention, the addition of EDTA or one of the known salts thereof, as stabiliser or complexing agent is unnecessary in the present formulation. Other embodiments may contain this compound or these compounds. In a preferred embodiment the content based on disodium EDTA is less than 100 mg/100 ml, preferably less than 50 mg/100 ml, more preferably less than 20 mg/100 ml. Generally, inhalable solutions in which the content of disodium EDTA is from 0 to 10 mg/100 ml are preferred.

According to the invention, the addition of ascorbic acid or one of the known salts thereof, as stabiliser or antioxidant is unnecessary in the present formulation. Other embodiments may contain this compound or these compounds. In a preferred embodiment the content based on ascorbic acid is less than 100 mg/100 ml, preferably less than 50 mg/100 ml, more preferably less than 20 mg/100 ml. Generally, inhalable solutions in which the content of ascorbic acid is from 0 to 10 mg/100 ml are preferred.

According to the invention, the addition of palmitate ester of ascorbic acid or one of the known salts thereof, as stabiliser or antioxidant is unnecessary in the present formulation. Other embodiments may contain this compound or these compounds. In a preferred embodiment the content based on palmitate ester of ascorbic acid is less than 100 mg/100 ml, preferably less than 50 mg/100 ml, more preferably less than 20 mg/100 ml. Generally, inhalable solutions in which the content of palmitate ester of ascorbic acid is from 0 to 10 mg/100 ml are preferred.

According to the invention, the addition of tocopherols or one of the known salts thereof, as stabiliser or antioxidant is unnecessary in the present formulation. Other embodiments may contain this compound or these compounds. In a preferred embodiment the content based on tocopherols is less than 100 mg/100 ml, preferably less than 50 mg/100 ml, more preferably less than 20 mg/100 ml. Generally, inhalable solutions in which the content of tocopherols is from 0 to 10 mg/100 ml are preferred.

According to the invention, the addition of lipoic acid or one of the known salts thereof, as stabiliser or antioxidant is unnecessary in the present formulation. Other embodiments may contain this compound or these compounds. In a preferred embodiment the content based on lipoic acid is less than 100 mg/100 ml, preferably less than 50 mg/100 ml, more preferably less than 20 mg/100 ml. Generally, inhalable solutions in which the content of lipoic acid is from 0 to 10 mg/100 ml are preferred. The lipoic acid may be in the racemic form or present as the (R) or (S) enantiomer.

Cosolvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin (asolectin), cocoa butter, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include physiologically acceptable salts such as sodium chloride as isotonic agents.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

/Preferred formulations contain, in addition to the solvent water and the combination of active substances cacao extract containing epicatechin and epicatechin oligomers, only benzalkonium chloride and disodium EDTA. In another preferred embodiment, no disodium EDTA is present. The propellant-free inhalable solutions according to the invention are administered in particular using inhalers of the kind which are capable of nebulising a small amount of a liquid formulation in the required therapeutic dose within a few seconds to produce an aerosol suitable for therapeutic inhalation. Within the scope of the present invention, preferred nebulisers are those in which a quantity of less than 100 microliters, preferably less than 50 microliters, more preferably between 20 and 30 microliters of active substance solution can be nebulised in preferably one spray action to form an aerosol with an average particle size of less than 20 micrometers, preferably less than 10 micrometers, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687.

The propellant-free inhalable solutions or suspensions according to the invention may take the form of concentrates or sterile inhalable solutions or suspensions ready for use, as well as the above-mentioned solutions and suspensions. Sterile formulations ready for use may be administered using energy-operated fixed or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods

### Fermentation Processes and Methods for Cacao to Enhance Epicatechin and Antioxidant Content

1. Fermentation of Cacao

Mature fruits (pods) rise directly from the stem of the cocoa tree and are thick walled and contain 30-40 beans (seeds). Each bean consists of two cotyledons and an embryo (radicle) surrounded by a seed coat (testa) and is enveloped in a sweet, white, mucilaginous pulp that comprises approximately 40% of seed fresh weight.

Cacao fermentation is one of the stages in post-harvest processing that governs ultimate product quality. Fermentation remains empirical and does not give rise to beans of consistent quality,

Fermentation helps to break down the mucilaginous pulp surrounding beans. It also helps to trigger biochemical changes inside the beans that contribute to reducing bitterness and astringency, and to the development of flavour precursors. Most of the work with fermentation processes has been in fact to maximize flavour of the final product.

There are several stages of the fermentation process. Generally, the cocoa pods, which are a fruit containing the beans within a pulp, are cut with machetes and placed into wooden boxes. In Africa, the pods are commonly simply thrown on a heap to ferment. Seeds within the ripe pod aremicrobiologically sterile. When the pod is opened with a knife, the pulp becomes contaminated with a variety of microorganisms many of which contribute to the subsequent fermentation. Organisms come mainly from the hands of workers, knives, unwashed baskets used for transport of seeds, and dried mucilage left on the walls of boxes from previous fermentations.

Cocoa pulp is a rich medium for microbial growth. It consists of 82-87% water, 10-15% sugar, 2-3% pentosans, 1-3% citric acid, and 1-1.5% pectin. Proteins, amino acids, vitamins (mainly vitamin C), and minerals are also present. The concentration of glucose, sucrose, and fructose is a function of fruit age. More glucose and fructose and a slight increase in total sugar concentration were observed in samples 6 days after harvest than in freshly harvested (ripe) pods.

During the first phase of fermentation, yeasts are highly active and alcoholic fermentation of the sugars which are present predominates. Oxygen is restricted to some degree, although the concept that the fermentation is truly anaerobic is probably fallacious. The yeast fermentation metabolism very quickly leads to consumption of all the simple sugars to give ethanol and carbon dioxide. Alcoholic fermentation is a moderately exothermic reaction (93.3 kJ by molecule of glucose consumed). It leads to a moderate increase in the temperature of the mass, which reaches 35 to 40° C. At the same time, polysaccharides in the cells of the mucilaginous tissue are broken down by the pectinolytic action of yeasts. The initial acidity of the pulp (pH 3.6), due to citric acid,

together with low oxygen levels, favor colonization by yeasts that are able to utilize pulp carbohydrates under both aerobic and anaerobic conditions. The size of the yeast population increases then remains almost constant for the next 12 h after which there is a dramatic decline of four orders of magnitude over the next day followed by a slower decrease leading to a final population of only a few viable cells per gram of pulp.

During the alcoholic phase of the fermentation a considerable amount of fluid drains out of the fermenting mass, when the fermentation is performed in wooden boxes. This is commonly called cacao “beer” and is consumed as palatable beverage. It can contain about 2-6% ethanol.

When fluid is drained away, the coverings of the boxes are partially removed, and the greater aeration of the mass due to the disappearance of the mucilage enables acetic and lactic acid bacteria to develop and intervene By oxidation, they convert the ethanol remaining which is produced during alcoholic (“anaerobic”) fermentation into acetic acid. Oxidation is a highly exothermic reaction (496 kJ per molecule of ethanol converted into acetic acid), which raises the temperature to 50° C. Regular stirring is necessary to promote aeration, so as to achieve quick and uniform fermentation, leading to a rapid increase in temperature. The lactic acid bacteria exhibit the fastest growth rate during the 16-48 h period of fermentation and are present in greater numbers, but not necessarily in biomass, than the yeasts for a short period of time. As aeration of the fermenting mass increases and the temperature rises above 37. C, acetic and lactic acid bacteria became the dominant organisms.

After the several-day fermentation process concludes, the beans are dried, typically in the sun with regular turning until the water content is less than 8%, which takes from one to four weeks. Alternatively, artificial dryers are used but it is important to keep the temperature not exceeding 60.0 and to dry slowly (at least 48 hours) during which time some excess acids may volatilize and some oxidation will occur, both of which are beneficial. The beans can then be stored for up to a year.

A certain amount of characterization has been performed of yeasts and bacteria which are present in cacao fermentation, See for example Ardhana, M M., Fleet, G H. (2003) “The microbial ecology of cocoa bean fermentations in Indonesia” International Journal of Food Microbiology 86 87-99; Camu, Nicholas, Tom De Winter, Kristof Verbrugghe, Ilse Cleenwerck, Peter Vandamme, Jemmy S. Takrama, Marc Vancanneyt, Luc De Vuyst (2007) “Dynamics and Biodiversity of Populations of Lactic Acid Bacteria and Acetic Acid Bacteria Involved in Spontaneous Heap Fermentation of Cocoa Beans in Ghana” Applied And Environmental Microbiology, 1809-1824;

Comi, G., Romano, P., Cocolin, L. Fiore, C. (2001) Characterization Of Kloeckera Apiculata Strains From The Friuli Region Of Northern Italy. World Journal of Microbiology and Biotechnology 17: 391-394; De Bruyne, Katrien, Nicholas Camu, Luc De Vuyst, Peter Vandamme (2009), “Lactobacillus fabifermentans sp. nov. and Lactobacillus cacaonum sp. nov., isolated from Ghanaian cocoa fermentations” International Journal of Systematic and Evolutionary Microbiology 59, 7-12; De Bruyne, Katrien, Camu, Nicholas, Lefebvre, Karen, De Vuyst Luc, Peter Vandamme (2008), “Weissella ghanensis sp. nov., isolated from a Ghanaian cocoa fermentation” International Journal of Systematic and Evolutionary Microbiology 58:2721-2725; De Vuyst L, Nicholas C, De Winter T, Vandemeulebroecke K, Vn de Perre V, Vancanneyt, M., De Vos, P., Cleenwerck I. “Validation of the (GTG)5-rep-PCR fingerprinting technique for rapid classification and identification of acetic acid bacteria, with a focus on isolates from Ghanaian fermented cocoa beans” International Journal of Food Microbiology 125 (2008) 79-90; Di Maro, E., Ercolini. D., Coppola, S. (2007) “Yeast dynamics during spontaneous wine fermentation of the Catalanesca grape” International Journal of Food Microbiology 117: 201-210; Galvez S L, Loiseau G, Paredes J L, Barel M Guiraud, J-P (2007) Study on the microflora and biochemistry of cocoa fermentation in the Dominican Republic International Journal of Food Microbiology 114: 124-130; Leal, G. A. Jr, Luiz Humberto Gomes, Priscilla Efraim, Flavio Cesar de Almeida Tavares Antonio Figueira (2008) “Fermentation of cacao (Theobroma cacao L.) seeds with a hybrid Kluyveromyces marxianus strain improved product quality attributes” FEMS Yeast Res 8 788-798; Lene Jespersen Dennis S, Nielsen, Susanne Honholt, Mogens Jakobsen (2005) “Occurrence and diversity of yeasts involved in fermentation of West African cocoa beans FEMS Yeast Research 5 441-453; Nielsen D. S. Teniola O. D Ban-Koffi., L., Owusu M., Andersson T. S., Holzapfel W. H. (2007) “The microbiology of Ghanaian cocoa fermentations analysed using culture-dependent and culture-independent methods” International Journal of Food Microbiology 114: 168-186; Osborn J P, and Edwards C G (2006), “Inhibition of malolactic fermentation by Saccharomyces during alcoholic fermentation under low- and high-nitrogen conditions: a study in synthetic media”, Australian Journal of Grape and Wine Research 12: 69-78; Romano, P, Fiore, C., Paraggio, M., Caruso, M., A. Capece (2003) “Function of yeast species and strains in wine flavor” International Journal of Food Microbiology 86 169-180; Ravelomanana, R. Guiraud J. P., Vincent J. C. Galzy P. (1985) ‘The yeast flora of cocoa bean fermentation in the Ivory Coast” MIRCEN Journal, 1, 319-326;
Sakharov Ivan Yu., Ardila Gerardo Bautista (1999) “Variations of peroxidase activity in cocoa (Theobroma cacao L.) beans during their ripening, fermentation and drying” Food Chemistry 65 51-54; which are incorporated by reference. Thus, many organisms have been identified as components of the flora, but these organisms have not been optimized. The few experiments which have been performed using inoculations with defined cultures have not focused upon epicatechin optimization.

We have discovered that this rather empirical process can be optimized for high concentration of epicatechins and procyanidins and epicatechin oligomers by the use of controlled fermentation rather than the empirical methods used today.

The invention accordingly comprises the processes involving the several steps and relation of one or more such steps with respect to each other, and the materials and products possessing the features, properties and relations of elements, all of which are exemplified in the detailed description, and the scope of the application, which will be indicated in the claims. We have discovered that cacao beans which are highly enriched in epicatechin and other polyphenolics antioxdiants can be produced by using controlled fermentation under modern conditions as opposed to uncontrolled fermentation

### EXAMPLES

### Example 1

### Mouse Ear Antivesicant Drug Screening Assay

The effect of topical application of HD to the medial aspect of the right ear was evaluated in albino male mice (CD-1 Strain, Charles Rivers Laboratory, Kingston, N.Y.) weighing about 25 to about 35 grams. Mice were weighed, marked for identification and anesthetized with a combination of ketamine (60 mg/kg) and xylazine (12 mg/kg) given as an intraperitoneal injection. In a fume hood, 5 [mgr]l (0.16 mg) of a 195 mM solution of neat (undiluted) HD (d[equals]1.27 g/ml; MW 159; purity 97.5%) in methylene chloride was applied to the medial surface of the right ear of each mouse using a digital microliter positive displacement pipette. This volume of HD allowed even distribution of the vesicant agent over the entire medial surface of the ear.

Mice were returned to polycarbonate cages for recovery, observation and treatment. Each cage was covered with a plastic backed paper diaper and warmed using a circulating water heating pad placed under the container. All animals were housed in the hood until euthanized in a halothane-filled chamber. The animals were euthanized 24 hours after exposure. Immediately after euthanasia, full thickness circular 8 mm punched specimens were taken from the center of each ear, placed into tarred 1.5 ml microfuge vials, and weighed to the nearest 0.1 mg on an analytical balance to determine tissue wet weight. This tissue wet-weight was used to determine an index of edema (relative ear weight, REW), which was used as the primary quantitative response to tissue injury. Each 8 mm punched biopsy specimen was then divided. One half of the tissue was placed into a vial comprising 10% neutral buffered formalin (NBF) for histopathological evaluation while the remaining half was snap frozen in liquid nitrogen for immunohistochemistry or for later processing in biochemical or molecular biology assays.

For each experiment, 10 mice per treatment group were used. The right ears of all groups of mice were exposed to HD liquid. Treatment groups were administered candidate antivesicant drugs as a pre-treatment (15 minutes before HD exposure) or post-treatment. Each mouse acted as its own control since the left ear was only treated with HD vehicle (MeCl2). In addition, 10 mice per experiment were used as HD positive controls. Previous in-house studies using a one-way analysis of variance (ANOVA) revealed no significant histopathologic differences in the effects of a methylene chloride (HD vehicle) treated ear and a control ear. Therefore, methylene chloride was only used on control ears that were used for biochemical or molecular biology assays.

After fixation in neutral buffered formalin (NBF), tissues were embedded in paraffin then sectioned and stained with hematoxylin/eosin (H&E) for microscopic evaluation. Histopathologic endpoints, subepidermal blister and epidermal necrosis, as described below, were given severity scores. See also Casillas, R. P., et al. (1997) Tox. Meth. 7:381-397 and Monteiro-Riviere, N. A., et al. (1999) J. Appl. Toxicol. 19, 313-328, both of which are herein incorporated by reference.

(a). Subepidermal blister (SEB; epidermal-dermal separation) and opposite side (contralateral) subepidermal blister (CSEB; unexposed outer surface). A SEB is any defect or discontinuity involving detachment of basal cells from the basement membrane.

(b). Epidermal necrosis (EN; exposed inner ear surface) and opposite side (contralateral) epidermal necrosis (CEN; unexposed outer ear surface). Epidermal necrosis denotes cellular death in the epithelium.

The severity scores were as follows: 0[equals]no lesion or change; 1[equals]change in less than 5% of the entire tissue section; 2[equals]change is present in 10%-40% of the entire tissue section; 3[equals]change is present in 50%-80% of the entire tissue section; 4[equals]change is present in greater than 90% of the entire tissue section. Scores were reported as the mean of each group.

Amount of irritant activity evidenced by bis(2-chloroethyl)sulfide (1% v/v in THF, 50 uL) applied to shaved skin of mouse graded visually after Rx with epicatechin oligomers. Vesicant was applied to shaved skin of mouse and 10 min later ointment was applied containing designated percentage of epicatechin oligomer extract in a PEG-300 base. PEG-300 base served as a control. Exemplary results are shown below:

| Dose of Epicatechin Oligomer Extract (w/w) | Grade |
|---|---|
| 0 | 5+ |
| 0.001% | 5-5+ |
| 0.01% | 5 |
| 0.1% | 4 |
| 0.5% | 3.5 |
| 1.0% | 2 |
| 5% | 1.5 |
| 10% | 1.5 |

### Example 2

### Murine Smoke Inhalation Screening Assay

We employed a murine model of smoke inhalation. Briefly, Adult female Swiss albino outbred mice were given a mix of ketamine (100 μg/g body wt), xylazine (5 μg/g body wt), and acepromazine (2.5 μg/g body wt) and placed in pairs into a smoke chamber for defined periods of wood smoke exposure. Untreated pine lumber (12×1×4 inches) was cut into small uniform rectangular pieces (20×3×3 mm) weighing a total of 0.1-0.5 g and placed between the nichrome wire heating coils in the smoke chamber. The wood was burned slowly so that it would not produce a flame. As the incinerator filled with smoke, a small fan in the chamber was turned on to circulate the smoke through the chamber with the anesthetized mice. After a defined period, the mice were removed, and the survivors were allowed to awaken from the anesthesia Control mice were also anesthetized and placed in the smoke chamber with intermittent exposure to air circulated by the fan but no smoke. In order to determine which material produced the greatest lung damage 48 or 72 h after a 12- to 15-min exposure, initial studies involved the pyrolysis of 10 mg/kg body wt of cotton, polyurethane, and wood (hardwood) to which female Swiss albino inbred mice anesthetized with chloral hydrate (0.1 ml/g body wt) were exposed.

Parameters were established which allowed the determination of a dose of smoke in a defined period of time which reproducibly produced a 50% mortality of the mice (LD₅₀ dose) In the study, 40 mice were exposed to the LD₅₀ dose of smoke. Half of the mice were pretreated by intracheal lavage with 100 micrograms of a mixture of epicatechin and epicatechin oligomers prepared from Theobroma Cacao, in Ringer's solution The other group of mice were treated by intratracheal lavage with Ringers solution alone. The results of the study are shown in the Table below.

TABLE 1

| Group | Mortality After 1 hour |
|---|---|
| Vehicle | 9/20 |
| Epicatechin | 1/20 |

These differences (total N = 40) were highly significant using Student's T test.

### Example 3

### Murine Smoke Inhalation Screening Assay: Larger Sample Size

Using similar methods to Example 1, a similar experiment was performed except that 80 mice were exposed to smoke at the LD₅₀ dos. Of these 80 mice, 44 immediately survived. Two groups (N=22 each) were formed. These surviving mice were treated after exposure to smoke with the intracheal lavage with 100 micrograms of a mixture of epicatechin and epicatechin oligomers prepared from Theobroma Cacao, in Ringer's solution (N=22) The rate of mortality of the mice over the following month was then compared with the rate of mortality of a comparable vehicle-treated group (N=22). This was done by a Kaplan-Meier analysis of a mortality curve for the treated and control groups. The expected deaths (calculated from the analysis of the untreated group) were then compared with the observed deaths in the treated group. This analysis showed a highly significant (p<0.005) reduction in excess deaths in the treated group. Detailed analysis of the Kaplan-Meier curves indicated that most of this difference was evidenced in the first ten-day period after smoke exposure.

### Example 4

### Fermentation of Cacao

The following example, illustrative of the present invention, employs a 200 L fermenter, which can be loaded with 50 Kg of cacao pod pulp. The fermenter, which was specially constructed for this work, is made of standard design of electropolished stainless steel, and is equipped with a jacket for heating with steam or cooling with water, a sealed gland joint for mechanical stirring, a port for the addition of oxygen, if required, a second port for exhaustion by a vacuum pump and addition of carbon dioxide, as required, another port for a sensor for the measurement of pO2, still another port for the insertion of a pH electrode, all the time while the fermenter remains sealed. It is also equipped with a pressure sensor to determine if excessive pressure builds up inside, and a pressure emergency release disk. A large valve at the bottom of the tank allows removal of fluid. The entire apparatus may be completely sterilized by heating with live steam, and my alternatively be sterilized by means of ethylene oxide gas or strong sodium hypochlorite solutions. Although this particular fermenter was of our design, it could be constructed by one normally skilled in the art, and does not differ substantially from such units such as are commercially marketed my commercial companies such as New Brunswick Scientific.

Into the fermenter is placed 50 Kg of pulp and beans obtained from cacao pods, obtained in a clean but not sterile manner. The material is agitated with a mechanical stirrer to break down the pulp and the material is innocuated with a culture of Saccharomyces cerevisiae (National Type Culture Collection). Sterile water (10 Kg) is added and the material maintained under carbon dioxide for a 3-day period at 37 C. At the end of this time, the alcoholic “beer” is removed from the fermenter through a stainless steel mesh under positive nitrogen pressure, allowing the pulp and beans which remain to be retained in said fermenter.

The liquid (20 Kg) which was removed was analyzed and found to contain 6.6% alcohol by volume and a low (0.6%) remaining concentration of glucose.

The fermenter was next inoculated with a lactic acid bacterium Lactobacillus brevis from NTCC, 20 Kg of sterile water containing 60 mM Phosphate and 50 mM of citrate, pH 3.3, was added, and the temperature raised to 37 C. Oxygen was added under monitoring from the pO2 probe to maintain a high constant value and acid production was monitored by pH.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising
a) a cacao extract, wherein the cacao extract comprises a mixture of epicatechin and one or more epicatechin oligomers; and
b) a pharmaceutically acceptable carrier.

2. The composition of claim 1, wherein the mixture is in a unit dosage form of about 0.01 to 10000 micrograms/g of cacao extract.

3. The composition of claim 1, wherein the pharmaceutically acceptable carrier comprises a monosaccharide, a disaccharide, a polysaccharide, a polyalcohol or a salt.

4. The composition of claim 1 wherein the composition is formulated for inhalation.

5. The composition of claim 4, wherein the formulation comprises an inhalable powder, propellant-containing metering aerosol or a propellant-free inhalable solution or suspension.

6. The composition of claim 5, wherein the inhalable powder comprises a monosaccharide, a disaccharide, a polysaccharide, a polyalcohol or a salt.

7. The composition of claim 1, wherein the cacao extract and/or the pharmaceutically acceptable carrier comprise a particle.

8. The composition of claim 7, wherein the particle is from about 10 to about 250 μmeters in size.

9. The composition of claim 8, wherein the particle is from about 10 to about 150 μmeters in size.

10. The composition of claim 8, wherein the particle is from about 15 to about 80 μmeters in size.

11. The composition of claim 1, wherein the pharmaceutically acceptable carrier comprises a lipid-based or polymer-based colloid.

12. The composition of claim 11, wherein the colloid is a liposome, a hydrogel, a microparticle, a nanoparticle or a block copolymer micelle.

13. The composition of claim 12, wherein the polymer-based colloid is a capsule.

14. A method of treating a subject who has an inflammatory or obstructive disease of the respiratory tract, the method comprising administering to the subject an effective amount of a composition a cacao extract, wherein the cacao extract comprises a mixture of epicatechin and one or more epicatechin oligomers; and a pharmaceutically acceptable carrier.

15. The method of claim 14, further comprising identifying a subject who has an inflammatory or obstructive disease of the respiratory tract.

16. The method of claim 14, wherein the subject is human.

17. The method of claim 14, wherein the inflammatory or obstructive disease of the respiratory tract is chronic obstructive pulmonary disease (COPD).

18. A method of treating a subject who has smoke-induced acute respiratory distress syndrome, the method comprising administering to the subject an effective amount of a composition a cacao extract, wherein the cacao extract comprises a mixture of epicatechin and one or more epicatechin oligomers; and a pharmaceutically acceptable carrier.

19. The method of claim 18, further comprising identifying a subject who has smoke-induced acute respiratory distress syndrome.

20. The method of claim 18, wherein the subject is human.

21. A method of treating a subject who has or who is at risk for an injury induced by a vesicant agent, the method comprising administering to the subject an effective amount of a composition comprising an epicatechin or an epicatechin oligomer and a pharmaceutically acceptable carrier.

22. The method of claim 21, further comprising identifying a subject who has or who is at risk for an injury induced by a vesicant agent.

23. The method of claim 21, wherein the epicatechin and epicatechin oligomers comprise an extract from the seed pods Theobroma Cacao.

24. The method of claim 21, further comprising administering an anti-vesicant agent.

25. The method of claim 24, wherein the anti-vesicant agent comprises 2-mercaptopyridine-N-oxide, 4-methyl-2-mercaptopyridine-N-oxide or 6-methyl-2-mercaptopyridine-N-oxide or a pharmaceutically acceptable salt thereof.

26. The method of claim 21, wherein the composition is administered before exposure to the vesicant agent.

27. The method of claim 21, wherein the composition is administered during exposure to the vesicant agent.

28. The method of claim 21, wherein the composition is administered after exposure to the vesicant agent.

29. The method of claim 21, wherein the vesicant agent is HD.

30. The method of claim 21, wherein the composition is administered topically, intraperitoneally, intravenously, subcutaneously, intramuscularly, transdermally, sublingually, or orally

31. A method for optimizing the yield of epicatechins in cacao fermentation, comprising (a) opening pods in a semisterile manner and placing in a stainless steel fermenter which has been previously sterilized; (b) partially homogenizing said pods in homogenizer using a mechanical agitator, (c) inoculating said fermenter containing said cacao pod homogenate with a yeast culture of a defined organism, (d) heating and cooling said fermenter, as necessary over a period of 24 hours to 96 hours whereby alcoholic fermentation takes place; (e) maintaining a constant value of pH, pO₂, and pCO₂ in the fermenter over this period, (f) removing fluid material from fermenter and retaining pulp and beans in fermenter, (g) introducing a culture of a lactic or acetic acid bacteria to the fermenter, (h) maintaining a constant value of pH, pO₂, and pCO₂ in the fermenter over this period, (i) removing acetic acid waste and collecting cacao beans following fermentation.

32. The method of claim 31, wherein the yeast is Saccharomyces cerevisiae.

33. The method of claim 31, wherein the yeast is S. cerevisiae var. chevalieri,

34. The method of 31, wherein the yeast is Candida bombi, Candida pelliculosa, Candida rugopelliculosa, Candida rugosa, Kloeckera apiculata, Kluyveromyces marxianus, Kluyveromyces thermotolerans, Lodderomyces elongisporus, Pischia Spp., Pichia fermentans, Torulaspora pretoriensis, or Saccharomyces pombe.

35. The method of claim 31, wherein the lactic acid bacteria is selected from Lactobacillus Acidophilus, Lactobacillus brevis, Lactobacillus casei, Lactobacillus Delbrueckii, Lactobacillus fermentum, Lactobacillus Lactis, Lactobacillus Plantarum, Lactococcus lactis, Leuconostoc mesenteroides, Pediococcus acidilactici, Pediococcus dextrinicum.

36. The method of claim 31, wherein the lactic acid bacteria is selected from Lactobacillus Acidophilus, Lactobacillus brevis, Lactobacillus casei, Lactobacillus Delbrueckii, Lactobacillus fermentum, Lactobacillus Lactis, Lactobacillus Plantarum, Lactococcus lactis, Leuconostoc mesenteroides, Pediococcus acidilactici, Pediococcus dextrinicum.

37. The method of claim 31, wherein the temperature of the alcoholic fermentation is maintained at a range from about 38° C. to about 50° C.

38. The method of claim 31, wherein the temperature of the alcoholic fermentation is maintained at a range from about 25° C. to about 37° C.

39. The method of claim 31, wherein the pH of the alcoholic fermentation is maintained at a range between about 3.0 to about 5.0.
